(19) United States
(12) Patent Application Publication
(10) Pub. No.: US 2011/0318393 A1
(43) Pub. Date: Dec. 29, 2011

(54) Substituted Pyrazole Compounds

(75) Inventors: Gaetan Ladouceur, Raleigh, NC (US); Gregory A. Agosto, Rockville, MD (US); Jamshed H. Shah, Silver Spring, MA (US); Lita Suwandi, Cary, NC (US); Nnamdi Ofoegbu, Cockeysville, MD (US)

(73) Assignee: MIIKANA THERAPEUTICS, INC., Rockville, MD (US)

(21) Appl. No.: 13/128,656

(22) PCT Filed: Nov. 23, 2009
(86) PCT No.: PCT/US09/65496

§ 371 Date: Aug. 3, 2011

Related U.S. Application Data
(60) Provisional application No. 61/118,136, filed on Nov. 26, 2008.

(51) Int. Cl. A61K 9/14; C07D 403/14; C07D 413/14; A61K 31/506; A61K 31/5377; C07D 405/14; A61P 29/00; A61P 25/00; A61P 35/00; A61P 9/00; A61P 37/08; A61P 11/06; A61P 5/00; A61P 35/02; A61P 17/10; A61P 31/04; A61P 31/22; A61P 27/06; A61P 33/02; A61P 19/02; A61P 7/06; A61P 17/00; A61P 17/06; C07D 401/14

(52) U.S. Cl. 424/400; 544/328; 544/295; 544/122; 514/210.2; 514/256; 514/252.18; 514/236.5; 514/252.19

(57) ABSTRACT

Provided are substituted pyrazole compounds which are useful as protein kinase inhibitors, compositions comprising the compounds, and methods of use thereof. The protein kinase inhibitors are particularly for inhibition of Aurora A (Aurora-2) protein kinase and are useful in the treatment of diseases associated with protein kinases, especially diseases associated with Aurora-2, such as cancer.

### CROSS REFERENCE TO RELATED APPLICATIONS

This application claims priority to U.S. Provisional Application No. 61/118,136, filed Nov. 26, 2008, the entirety of which is incorporated herein.

### FIELD OF THE INVENTION

This invention is directed to protein kinase inhibitors, compositions comprising such inhibitors, and methods of use thereof. More particularly, the invention relates to inhibitors of Aurora A (Aurora-2) protein kinase. The invention also relates to pharmaceutical compositions, as well as to methods of treating diseases associated with protein kinases, especially diseases associated with Aurora A, such as cancer.

### BACKGROUND OF THE INVENTION

The search for new therapeutic agents has been greatly aided in recent years by better understanding of the structure of enzymes and other biomolecules associated with target diseases. One important class of enzymes that has been the subject of extensive study is the protein kinases.

Protein kinases mediate intracellular signal transduction by effecting a phosphoryl transfer from a nucleoside triphosphate to a protein acceptor that is involved in a signaling pathway. There are a number of kinases and pathways through which extracellular and other stimuli cause a variety of cellular responses to occur inside the cell. Examples of such stimuli include environmental and chemical stress signals (e.g. osmotic shock, heat shock, ultraviolet radiation, bacterial endotoxin, H₂O₂), cytokines (e.g. interleukin-1 (IL-1) and tumor necrosis factor alpha (TNF-alpha)), and growth factors (e.g. granulocyte macrophage-colony-stimulating factor (GM-CSF), and fibroblast growth factor (FGF). An extracellular stimulus may effect one or more cellular responses related to cell growth, migration, differentiation, secretion of hormones, activation of transcription factors, muscle contraction, glucose metabolism, control of protein synthesis and regulation of cell cycle.

Many diseases are associated with abnormal cellular responses triggered by protein kinase-mediated events. These diseases include autoimmune diseases, inflammatory diseases, neurological and neurodegenerative diseases, cancer, cardiovascular diseases, allergies and asthma, Alzheimer's disease or hormone-related diseases. Accordingly, there has been a substantial effort in medicinal chemistry to find protein kinase inhibitors that are effective as therapeutic agents.

In humans, there are three highly related Aurora kinases that are all serine/threonine protein kinases (see Andrews, P. D., et al., Curr. Opin. Cell. Biol. 2003, 15, 672-683; Carmena, M., Earnshaw, W. C., Nat. Rev. Mol. Cell. Biol. 2003, 4, 842-854; Brown, J. R., et al., BMC Evol. Biol. 2004, 4, 39, Andrews, P. D., Oncogene 2005, 24, 5005-5015). Despite the sequence relatedness of Aurora A, B and C, the localization and function of these kinases is quite distinct. As a result, overexpression or activation of each of these kinases can be associated with different disease states, including proliferative diseases such as cancer.

Members of the family demonstrate distinct subcellular localization during mitosis and are degraded by the proteosome following exit from mitosis (Graham et al. (2002) J. Biol. Chem. 277:42419-22). The kinases are often found complexed with other proteins, including cytoskeletal structures.

The Aurora kinases share a conserved C-terminal catalytic domain, with greater variation being observed at the N-terminus. Aurora A (Aurora-2) is unique in the presence of two lysine residues in the nucleotide-binding domain of the kinase (Warner et al. (2003) Molecular Cancer Therapeutics 2:589-95).

Maximum levels of the Aurora A polypeptide, and maximum Aurora A activity, are observed at the G₂/M transition leading into mitotic prophase, with the polypeptide localizing to the mitotic spindle pole (Graham et al. (2002) J. Biol. Chem. 277:42419-22; Sakai et al. (2002) J. Biol. Chem. 277:48714-23). Aurora A appears to regulate chromosome duplication with aberrant expression being associated with aneuploidy and an aggressive clinical phenotype, particularly in solid tumors. Such observations, and additional experimental data, suggest that Aurora A disrupts the signaling cascade that regulates chromosome segregation (Sen et al. (2002) J. Nat. Cancer. Inst. 94:1320-29).

Aurora A also appears to function in meiosis, likely in separating homologous chromosomes and in spindle rotation. Injection of antibodies against Aurora A into Xenopus oocytes prevents first polar body extrusion and causes arrest at meiosis I (Castro et al. (2003) J. Biol. Chem. 2236-41). The Xenopus kinesin-like protein, Eg5, is known to be a substrate for Aurora-2 (Castro et al. (2003) J. Biol. Chem. 2236-41).

In addition, in vitro studies show that Aurora A is incorporated into chromatin and phosphorylates histone H3, and possibly histone H2B (Scrittori et al. (2001) J. Biol. Chem. 276:30002-10). H3 phosphorylation, e.g., at serine-10, during chromosome assembly, appears to be a conserved event in eukaryotic cell division. Inhibition of H3 phosphorylation leads to chromosome condensation, abnormal segregation, and the loss of chromosomes during mitosis and meiosis (Scrittori et al. (2001) J. Biol. Chem. 276:30002-10).

Accordingly, the emerging model for histone phosphorylation is analogous to that of histone acetylation, wherein partially redundant enzymatic activities are associated with histone modifications but different enzymes may function in different cellular contexts. For example, some enzymes may modify histones in bulk, while other enzymes modify histones in a targeted manner, i.e., in a sequence or domain-specific manner in the context of assembled chromatin (see, e.g., Scrittori et al. (2001) J. Biol. Chem. 276:30002-10). According to this model, Aurora A would appear to be a kinase responsible for targeted histone modification, in the context of assembled or assembling chromatin.

Other members of the Aurora kinase family are also associated with mitosis and meiosis. Aurora B, like Aurora A, is involved in distinct protein phosphorylation events that regulate the cell cycle. Unlike Aurora A, Aurora B is localized to inner-centromeric chromatin from prophase until the metaphase-anaphase transition, relocalizes to the microtubules in the spindle midzone during telophase, and subsequently is found in the midbody throughout cytokinesis (See Andrews, P. D., Oncogene 2005, 24, 5005-5015, loc. cit.). The function of Aurora B is to ensure accurate chromosome segregation and appropriate cytokinesis. Aurora B appears to associate with a survivin, a polypeptide that associates with the inner centromere and undergoes a significant degree of stretching during mitosis. Survivin appears to be involved with inhibition of apoptosis as well as cell cycle control. Interestingly, both Aurora B and survivin are delocalized during megakaryocyte endomitosis, a process by which late anaphase and cytokinesis are skipped, leading to megakaryocyte polyploidy (Zhang et al. (2004) Blood 103:3717-26). Inhibitors of this function in a proliferative disease, such as cancer, would lead to stasis and cell death, making such inhibitors useful in cancer chemotherapy.

Aurora C (Aurora-3) is the least studied, known member of the family. Aurora C localizes to centrosomes from anaphase until telophase (or even cytokinesis), and is highly expressed in the testis (Brown et al. (2004) BMC Evolutionary Biology 4:39).

As noted above, Aurora kinases are overexpressed in certain types of cancers, including colon, breast, and other solid-tumor cancers. The genes encoding the Aurora B and A kinases tend to be amplified in certain types of cancers, while the gene encoding the Aurora C kinase resides in a region of the chromosome that is subject to rearrangement and deletion. Aurora A has been associated with a variety of malignancies, including primary colon, colorectal, breast, stomach, ovarian, prostate, and cervical cancer, neuroblastoma, and other solid-tumor cancers (Warner et al. (2003) Molecular Cancer Therapeutics 2:589-95).

Inhibitors of Aurora A have been described. For example, Harrington et al. ((2004) Nat. Med. 10:262-67) have described VX-680, a small-molecule inhibitor that blocks cell-cycle progression and induces apoptosis in certain types of tumors in in vivo xenograft models. A pyrazole Aurora A kinase inhibitor is also described in U.S. Pat. No. 6,653,301 (Bebbington et al., issued Nov. 25, 2003).

Hauf et al. ((2003) J. Cell. Biol. 161:281-294) identified the indolinone (Hesperadin) as an inhibitor of Aurora B, which causes cells to enter anaphase with monooriented chromosomes, having both sister kinetochores attached to a single spindle pole (a condition known as syntelic attachment).

Ditchfield et al. ((2003) J. Cell. Biol. 161:267-280) described ZM447439 ((4-(4-(N-benzoylamino)anilino)-6-methoxy-7-(3-(1-morpholino)propoxy)quina-zoline), an Aurora kinase inhibitor which interferes with chromosome alignment, segregation, and cytokinesis.

Accordingly, kinase inhibitors, particularly inhibitors of Aurora kinases, are of particular interest in treating certain disorders, including cancer. Compounds exhibiting such inhibition are of particular value.

### SUMMARY OF THE INVENTION

The present invention provides compounds or pharmaceutically acceptable derivatives or prodrugs thereof, compositions, and methods for treating diseases mediated by kinases. Such diseases include primary, secondary, and metastatic cancers such as melanoma, lymphoma, leukemia, colon, colorectal, breast, lung, kidney, pancreatic, renal, CNS, stomach, ovarian, prostate, cervical, and neuroblastoma.

In a first embodiment, the invention provides a compound of the Formula I:

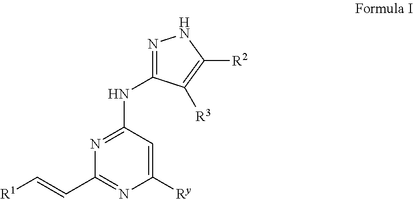

or a pharmaceutically acceptable derivative or prodrug thereof, wherein:

R^{y} is

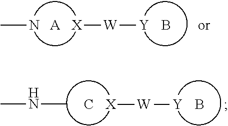

X is N, C or CR;

Y is N, C, or CR;

Ring A is an optionally substituted 4, 5 or 6 membered monocyclic heterocyclic ring;

Ring B is an optionally substituted 3-7 membered monocyclic or 8-10 membered bicyclic ring selected from carbocyclyl, aryl, heterocyclyl, or heteroaryl;

Ring C is an optionally substituted 3, 4, 5 or 6 membered heterocyclic, carbocyclic, aryl or heteroaryl ring;

W is (CR₂)ₙ, where n is 0, 1, 2, 3, 4 or 5;

R¹ is an optionally substituted monocyclic or bicyclic aryl ring;

R² and R³ are independently hydrogen, optionally substituted aliphatic, optionally substituted carbocyclyl, optionally substituted aryl, optionally substituted heteroaryl, optionally substituted heterocyclyl, or R² and R³ taken together with their intervening atoms form a fused, optionally substituted unsaturated or partially unsaturated ring having 0-3 ring heteroatoms; and

R is hydrogen, aliphatic, aryl, aralkyl, alkylaryl, heterocyclyl, heteroaryl, halo, hydroxyl, alkoxy, amino, alkylamino, or dialkylamino.

In another embodiment, a compound of Formula I is provided, wherein R² is aliphatic. In another embodiment, a compound of Formula I is provided wherein R¹ is phenyl.

In certain embodiments, W is a bond or n is 0. In other embodiments, W is —CH₂—. In other embodiments, W is —CH₂CH₂—. In one embodiment, n is 1. In one embodiment, n is 2.

In one embodiment, R is hydrogen.

In other embodiments of the invention, a compound of Formula I is provided wherein R¹ is phenyl, R² is aliphatic, R³ is H, and R^{y} has the structure

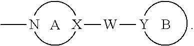

In still another embodiment of the invention, a compound of Formula I is provided, wherein R¹ is phenyl, R² is aliphatic, R³ is H, and R^{y} has the structure

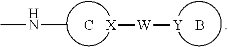

In another embodiment of Formula I, R¹ is phenyl, R² is aliphatic, R³ is H, R^{y} has the structure

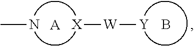

Ring A is a 4, 5 or 6 membered ring, and Ring B is aryl, heterocyclyl, or heteroaryl.

In another embodiment of Formula I, R¹ is phenyl, R² is aliphatic, R³ is H, R^{y} is

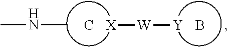

Ring C is heterocyclyl or aryl and Ring B is aryl, heteroaryl, or heterocyclyl.

In various embodiments of Formula I, including those described above, Ring B is optionally substituted phenyl ring. In other embodiments of Formula I, Ring B is optionally substituted pyridinyl, optionally substituted piperidinyl, optionally substituted pyrrolidinyl, optionally substituted pyridinyl, or optionally substituted morpholinyl.

In still other embodiments of Formula I, a compound is provided wherein R¹ is phenyl, R² is aliphatic, R³ is H, R^{y} is

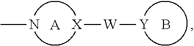

Ring A is a 4, 5, or 6 membered ring, Ring B is aryl, carbocyclyl or heterocyclyl, X is CR, W is (CH₂)ₙ, where n is 0, 1, or 2, and R is hydroxyl.

In another embodiment, a compound of Formula I is provided wherein R¹ is phenyl, R² is aliphatic, R³ is H, R^{y} is

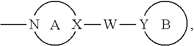

Ring A is a 4, 5 or 6 membered ring, X is N, and Ring B is aryl, heteroaryl, or heterocyclyl.

In another aspect of the invention, a compound of Formula Ia is provided:

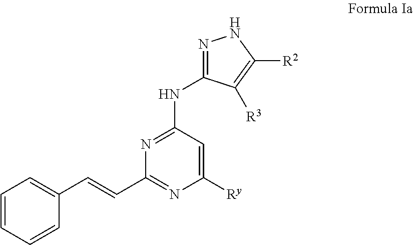

wherein variables R², R³ and R^{y} are as defined for Formula I above.

In still another aspect of the invention, a compound of Formula Ib is provided:

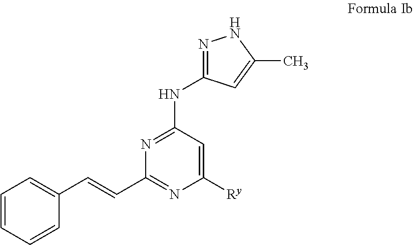

where variable R^{y} is as defined for the compound of Formula I described above.

In one embodiment of Formula Ib, a compound is provided wherein R^{y} is

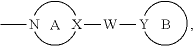

Ring A is a 4, 5 or 6 membered ring, X is CR, W is (CH₂)ₙ, where n is 0, 1, or 2, and Ring B is phenyl, piperidinyl, morpholinyl, pyridinyl, pyrimidinyl, or homopiperidinyl.

In another embodiment of Formula Ib, a compound is provided wherein R^{y} is

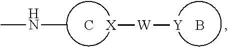

Ring C is phenyl or heterocyclyl, W is (CH₂)ₙ, where n is 0, 1, or 2, and Ring B is phenyl, heterocyclyl or heteroaryl.

In particular embodiments, the invention provides compounds with the structures provided in Table 1 shown below. Biologically acceptable salts and prodrugs of the compounds are also provided.

In another aspect of the invention, pharmaceutical compositions comprising an Aurora kinase A inhibition effective amount of the compound of Formulae I, Ia or Ib, in combination with a pharmaceutically acceptable carrier, adjuvant or vehicle are provided.

In some embodiments, the pharmaceutical compositions comprise particles that are less than about 2 microns average particle size. In other embodiments the compositions are incorporated into a biodegradable or non-biodegradable polymer.

In still another embodiment, the compositions comprise the compounds of Formulae I, Ia or Ib and an additive. The additive may be an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

In another embodiment of the invention, the pharmaceutical compositions comprise a carrier that is suitable for oral, parenteral, intraveneous, subcutaneous, inhalation, topical, or intradermal administration.

In another aspect of the invention, a method of treating a patient with a disease comprising administering to the patient with the disease an effective amount of a compound of Formulae I, Ia or Ib is provided, wherein the disease is an autoimmune disease, inflammatory disease, neurological or neurodegenerative disease, cancer, cardiovascular disease, allergy, asthma, or a hormone-related disease.

In one embodiment, the invention provides a method of treating a patient with a cancer comprising administering to the patient having the cancer an effective cancer-treating amount of a compound of Formulae I, Ia or Ib. In some embodiments, the cancer is a solid tumor, blood borne tumor, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity, pharynx, lip, tongue, mouth, pharynx, small intestine, colon-rectum, large intestine, rectum, brain and central nervous system, or leukemia.

In another aspect of the invention, a method of treating a patient with a disease associated with undesirable neovascularization is provided, wherein the method comprises administering to the patient with the undesirable neovascularization an effective amount of a compound of Formulae I, Ia or Ib. In various embodiments, the disease associated with undesirable neovascularization comprises ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, or corneal graph rejection, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, or post-laser complications.

In still another aspect of the invention, a method of treating a patient with an inflammatory disease associated with inflammation is provided, wherein the method comprises administering to the patient with the inflammatory disease an effective amount of a compound of Formulae I, Ia or Ib. In some embodiments, the inflammatory disease is excessive or abnormal stimulation of endothelial cells, atherosclerosis, vascular malfunctions, abnormal wound healing, inflammatory and immune disorders, Bechet's disease, gout or gouty arthritis, abnormal angiogenesis accompanying rheumatoid arthritis, skin diseases, psoriasis, diabetic retinopathy, retinopathy of prematurity, retrolental fibroplasia, macular degeneration, corneal graft rejection, neovascular glaucoma or Osler Weber syndrome.

In other embodiments of the invention, the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

These and other objects, features and advantages of the present invention will become apparent after a review of the following detailed description of the disclosed embodiments and the appended claims.

### DETAILED DESCRIPTION

Terms used herein will have their customary meaning in the art unless indicated otherwise. The phrase “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

The term “aliphatic” or “alkyl” as used herein means straight-chain, branched or cyclic C₁-C₁₄ hydrocarbons which are completely saturated or which contain one or more units of unsaturation, but which are not aromatic. For example, suitable aliphatic groups include substituted or unsubstituted linear, branched or cyclic alkyl, alkenyl, alkynyl groups, or hybrids thereof, such as (cycloalkyl)alkyl, (cycloalkenyl)alkyl, or (cycloalkyl)alkenyl. The terms “alkoxy”, “hydroxyalkyl”, “alkoxyalkyl”, and “alkoxycarbonyl”, used alone or as part of a larger moiety includes both straight and branched chains containing one to fourteen carbon atoms. Illustrative examples of alkyl groups are methyl, ethyl, propyl, isopropyl, cyclopropyl, butyl, secbutyl, isobutyl, tertbutyl, cyclobutyl, 1-methylbutyl, 1,1-dimethylpropyl, pentyl, cyclopentyl, isopentyl, neopentyl, cyclopentyl, hexyl, isohexyl, and cyclohexyl. Unless otherwise specified, the alkyl group can be unsubstituted or substituted with one or more moieties selected from the group consisting of alkyl, halo, haloalkyl, hydroxyl, carboxyl, acyl, acyloxy, amino, amido, carboxyl derivatives, alkylamino, dialkylamino, arylamino, alkoxy, aryloxy, nitro, cyano, thio, sulfonyl, ester, carboxylic acid, amide, phosphonyl, phosphinyl, thioether, oxime, or any other viable functional group that does not inhibit the pharmacological activity of this compound, either unprotected, or protected as necessary, as known to those skilled in the art, for example, as taught in Greene, et al., Protective Groups in Organic Synthesis, John Wiley and Sons, Second Edition, 1991.

The terms “alkenyl” and “alkynyl” used alone or as part of a larger moiety shall include both straight and branched chains containing two to fourteen carbon atoms. An “alkenyl” group as used herein means straight-chain, branched or cyclic C₁-C₁₄ hydrocarbons which are include one or more units of unsaturation, comprising at least one double carbon-carbon bond. Suitable alkenyl groups include, but are not limited to (C₂-C₈)alkenyl groups, such as ethenyl, propenyl, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An “alkynyl” group as used herein means straight-chain, branched or cyclic C₁-C₁₄ hydrocarbons which are include two or more units of unsaturation, comprising at least one triple carbon-carbon bond.

The term “amino” refers to an NH₂ group.

The term “alkylamino” refers to an amino group wherein one of the hydrogen atoms is replaced by an alkyl group.

The teen “dialkylamino” refers to an amino group wherein the hydrogen atoms are replaced by alkyl groups, wherein the alkyl group may be the same or different.

The terms “haloalkyl”, “haloalkenyl” and “haloalkoxy” means alkyl, alkenyl or alkoxy, as the case may be, substituted with one or more halogen atoms.

The term “halogen” means F, Cl, Br, or I.

The term “heteroatom” means nitrogen, oxygen, or sulfur and includes any oxidized form of nitrogen and sulfur, and the quaternized form of any basic nitrogen. Also the term “nitrogen” includes a substitutable nitrogen of a heterocyclic ring. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl) or NR⁺ (as in N-substituted pyrrolidinyl).

The terms “carbocycle”, “carbocyclyl”, “carbocyclo”, “carbocyclic”, or “cycloalkyl” used alone or as part of a larger moiety shall include cyclic C₃-C₁₄ hydrocarbons, or aliphatic ring systems of 3 to 14 members, which are saturated or which contain one or more units of unsaturation, but which are not aromatic. In particular, the ring may contain one or more double bonds, but are not aromatic. The terms “carbocycle”, “carbocyclyl”, “carbocyclo”, or “carbocyclic” whether saturated or partially unsaturated, also refers to rings that are optionally substituted. The terms “carbocycle”, “carbocyclyl”, “carbocyclo”, or “carbocyclic” also include aliphatic rings that are fused to one or more aromatic or nonaromatic rings, such as in a decahydronaphthyl or tetrahydronaphthyl, where the radical or point of attachment is on the aliphatic ring.

The term “aryl” used alone or as part of a larger moiety as in “aralkyl”, “aralkoxy”, or “aryloxyalkyl”, refers to carbon-based aromatic ring groups having six to fourteen members, such as phenyl, benzyl, phenethyl, 1-naphthyl, 2-naphthyl, 1-anthracyl and 2-anthracyl. The term “aryl” also refers to rings that are optionally substituted. The term “aryl” may be used interchangeably with the term “aryl ring”. “Aryl” also includes fused polycyclic aromatic ring systems in which an aromatic ring is fused to one or more rings. Examples include 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in an indanyl, phenanthridinyl, or tetrahydronaphthyl, where the radical or point of attachment is on the aromatic ring.

The term “aralkyl,” unless otherwise specified, refers to an aryl group as defined above linked to the molecule through an alkyl group as defined above.

The term “alkaryl,” unless otherwise specified, refers to an alkyl group as defined above linked to the molecule through an aryl group as defined above. Other groups, such as acyloxyalkyl, alkoxyalkyl, alkoxycarbonyl, alkoxycarbonylalkyl, alkylaminoalkyl, alkylthioalkyl, amidoalkyl, aminoalkyl, carboxyalkyl, dialkylaminoalkyl, haloalkyl, heteroaralkyl, heterocyclicalkyl, hydroxyalkyl, sulfonamidoalkyl, sulfonylalkyl and thioalkyl are named in a similar manner.

The term “alkoxy,” unless otherwise specified, refers to a moiety of the structure —O— alkyl, wherein alkyl is as defined above.

The term “acyl,” refers to a group of the formula C(O)R′ or “alkyl-oxy”, wherein R′ is an alkyl, aryl, alkaryl or aralkyl group, or substituted alkyl, aryl, aralkyl or alkaryl.

The term “heterocycle”, “heterocyclyl”, or “heterocyclic” as used herein includes non-aromatic ring systems having three to fourteen members, preferably four to ten, in which one or more ring carbons, preferably one to four, are each replaced by a heteroatom. Examples of heterocyclic rings include 3-1H-benzimidazol-2-one, (1-substituted)-2-oxo-benzimidazol-3-yl, 2-tetrahydro-furanyl, 3-tetrahydrofuranyl, 2-tetrahydropyranyl, 3-tetrahydropyranyl, 4-tetra-hydropyranyl, [1,3]-dioxalanyl, [1,3]-dithiolanyl, [1,3]-dioxanyl, 2-tetra-hydro-thiophenyl, 3-tetrahydrothiophenyl, morpholinyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, homopiperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

The term “heteroaryl”, used alone or as part of a larger moiety as in “heteroaralkyl” or “heteroarylalkoxy”, refers to heteroaromatic ring groups having five to fourteen members. Examples of heteroaryl rings include 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, pyridinyl or pyridyl, 2-pyridyl, 3-pyridyl, 4-pyridyl, pyrimidinyl or pyrimidyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, and benzoisoxazolyl. Also included within the scope of the term “heteroaryl”, as it is used herein, is a group in which a heteroatomic ring is fused to one or more aromatic or nonaromatic rings where the radical or point of attachment is on the heteroaromatic ring. Examples include tetrahydroquinolinyl, tetrahydroisoquino-linyl, and pyrido [3,4-d]pyrimidinyl. The term “heteroaryl” also refers to rings that are optionally substituted. The term “heteroaryl” may be used interchangeably with the term “heteroaryl ring” or the term “heteroaromatic”.

An aryl (including aralkyl, aralkoxy, aryloxyalkyl and the like) or heteroaryl (including heteroaralkyl and heteroarylalkoxy and the like) group may contain one or more substituents. Examples of suitable substituents on any unsaturated carbon atom of an aryl, heteroaryl, aralkyl, or heteroaralkyl group include a halogen, CF₃, —R⁰, —OR⁰, —SR⁰, 1,2-methylene-dioxy, 1,2-ethylenedioxy, protected OH (such as acyloxy), phenyl (Ph), substituted Ph, —O(Ph), substituted —O(Ph), —CH₂(Ph), substituted —CH₂(Ph), —CH₂CH₂(Ph), substituted —CH₂CH₂(Ph), —NO₂, —CN, —N(R⁰)₂, —NR⁰C(O)R⁰, —NR⁰C(O)N(R⁰)₂, —NR⁰CO₂R⁰, —NR⁰NR⁰C(O)R⁰, —NR⁰NR⁰C(O)N(R⁰)₂, —NR⁰NR⁰C₂R⁰, —C(O)C(O)R⁰, —C(O)CH₂C(O)R⁰, —CO₂R⁰, —C(O)R⁰, —C(O)N(R⁰)₂, —OC(O)N(R⁰)₂, —S(O)₂R⁰, —SO₂N(R⁰)₂, —S(O)R⁰, —NR⁰SO₂N(R⁰)₂, —NR⁰SO₂R⁰, —C(═S)N(R⁰)₂, —C(═NH)—N(R⁰)₂, —(CH₂)_{y}NHC(O)R⁰, and —(CH₂)_{y}NHC(O)CH(V—R⁰)(R⁰); wherein each R⁰ is independently selected from hydrogen, a substituted or unsubstituted aliphatic group, an unsubstituted heteroaryl or heterocyclic ring, phenyl (Ph), substituted Ph, —O(Ph), substituted —O(Ph), —CH₂(Ph), or substituted —CH₂(Ph); y is 0-6; and V is a linker group. Examples of substituents on the aliphatic group or the phenyl ring of R⁰ include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

An aliphatic group or a non-aromatic heterocyclic ring or a fused aryl or heteroaryl ring may contain one or more substituents. Examples of suitable substituents on any saturated carbon of an aliphatic group or of a non-aromatic heterocyclic ring or a fused aryl or heteroaryl ring include those listed above for the unsaturated carbon of an aryl or heteroaryl group and the following: ═O, ═S, ═NNHR*, ═NN(R*)₂, ═N—, ═NNHC(O)R*, ═NNHCO₂(alkyl), ═NNHSO₂ (alkyl), or ═NR*, where each R* is independently selected from hydrogen, an unsubstituted aliphatic group, or a substituted aliphatic group. Examples of substituents on the aliphatic group include amino, alkylamino, dialkylamino, aminocarbonyl, halogen, alkyl, CF₃, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

Suitable substituents on the nitrogen of a non-aromatic heterocyclic ring include —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, and —NR⁺SO₂R⁺; wherein each R⁺ is independently selected from hydrogen, an aliphatic group, a substituted aliphatic group, phenyl (Ph), substituted Ph, —O(Ph), substituted —O(Ph), —CH₂(Ph), substituted —CH₂(Ph), —CH(Ph)₃ or an unsubstituted heteroaryl or heterocyclic ring. Examples of substituents on the aliphatic group or the phenyl ring include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

The term “linker group” or “linker” means an organic moiety that connects two parts of a compound. Linkers are typically comprised of an atom such as oxygen or sulfur, a unit such as —NH—, —CH₂—, —C(O)—, —C(O)NH—, or a chain of atoms, such as an alkylidene chain. The molecular mass of a linker is typically in the range of about 14 to 200, preferably in the range of 14 to 96 with a length of up to about six atoms. Examples of linkers include a saturated or unsaturated C₁₋₆ alkylidene chain which is optionally substituted, and wherein one or two saturated carbons of the chain are optionally replaced by —C(O)—, —C(O)C(O)—, —CONH—, —CONHNH—, —CO₂—, —OC(O)—, —NHCO₂—, —O—, —NHCONH—, —OC(O)NH—, —NHNH—, —NHCO—, —S—, —SO—, —SO₂—, —NH—, —SO₂NH—, or —NHSO₂—.

The term “alkylidene chain” refers to an optionally substituted, straight or branched carbon chain, that may be fully saturated or have one or more units of unsaturation. The optional substituents are as described above for an aliphatic group.

A combination of substituents or variables is permissible only if such a combination results in a stable or chemically feasible compound. A stable compound or chemically feasible compound is one in which the chemical structure is not substantially altered when kept in the dark at a temperature of 40° C. or less, in the absence of moisture or other chemically reactive conditions, for at least a week.

It is understood that in all substituted groups defined herein, polymers arrived at by defining substituents with further substituents to themselves (e.g., substituted phenyl having a substituted phenyl as a substituent which is itself substituted with a substituted phenyl, etc.) are not intended for inclusion herein. In such cases, the maximum number of such substituents is three. For example, phenyl substituted with a substituted phenyl is limited to -substituted phenyl-(substituted phenyl)-(substituted phenyl).

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention. Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are within the scope of this invention.

Whenever a range is referred to herein, it includes independently and separately every member of the range. As a non-limiting example, the term “C₁-C₁₀ alkyl” is considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities. Similarly, as another non-limiting example, 1-10% includes independently, 1%, 2%, 3%, 4%, 5%, 6%, 7%, 8%, 9% and 10%, as well as ranges in between such as 1-2%, 2-3%, etc.

Compounds of Formulae I, Ia or Ib, or salts thereof, may be formulated into compositions. In one embodiment, the composition is a pharmaceutical composition. In one embodiment, the composition comprises an amount of the protein kinase inhibitor effective to inhibit a protein kinase in a biological sample or in a patient. Compounds of this invention and phatinaceutical compositions thereof, which comprise an amount of the protein kinase inhibitor effective to treat or prevent a kinase mediated condition and a pharmaceutically acceptable carrier, adjuvant, or vehicle, may be formulated for administration to a patient.

Another aspect of this invention relates to a method of treating or preventing a kinase mediated disease. In one embodiment, the disease is a Aurora A-mediated disease, which method comprises administering to a patient in need of such a treatment a therapeutically effective amount of a compound of Formulae I, Ia or Ib, or a pharmaceutical composition thereof.

The term “Aurora A-mediated disease” or “Aurora A-mediated condition”, as used herein, means any disease or other deleterious condition in which Aurora is thought to play a role. The terms “Aurora A-mediated disease” or “Aurora A-mediated condition” also mean those diseases or conditions that are alleviated by treatment with an Aurora A inhibitor. Such conditions include cancer.

The term “cancer” includes, but is not limited to, solid tumors and blood borne tumors and include, but is not limited to, the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia. The term “cancer” includes primary cancer, cancers secondary to treatment, and metastatic cancers.

An aspect of the invention relates to compounds and compositions that are useful for treating cancer.

Another aspect of the invention relates to the treatment of the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia.

Another aspect of the invention is a method for treating cancer comprising administering one or more of the compounds described herein to a patient with cancer.

Angiogenesis is characterized by the proliferation of endothelial cells to form new blood vessels (often called neovascularization). Inhibition of mitosis of endothelial cells results in inhibition of angiogenesis. Another aspect of this invention therefore relates to inhibition of undesirable mitosis, including undesirable angiogenesis. A mammalian disease characterized by undesirable cell mitosis, as defined herein, includes, but is not limited to, excessive or abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying rheumatoid arthritis, skin diseases, such as psoriasis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease).

Other undesired angiogenesis involves normal processes including ovulation and implantation of a blastula. The compositions described above can be used as a birth control agent by reducing or preventing uterine vascularization required for embryo implantation. Accordingly, the compositions described above can be used to block ovulation and implantation of a blastula or to block menstruation (induce amenorrhea).

Diseases associated with undesirable mitosis, including neovascularization, can be treated according to the present invention. Such diseases include, but are not limited to, ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, and corneal graph rejection.

Other diseases associated with undesirable mitosis, including neovascularization, can be treated according to the present invention. Such diseases include, but are not limited to, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, and post-laser complications. Other diseases include, but are not limited to, diseases associated with rubeosis (neovascularization of the iris and the angle) and diseases caused by the abnormal proliferation of fibrovascular or fibrous tissue including all forms of proliferative vitreoretinopathy, whether or not associated with diabetes.

Another aspect of the invention relates to the treatment of inflammatory diseases including, but not limited to, excessive or abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying rheumatoid arthritis, skin diseases, such as psoriasis, diabetic retinopathy and other ocular angiogenic diseases such as retinopathy of prematurity (retrolental fibroplasic), macular degeneration, corneal graft rejection, neovascular glaucoma and Osler Weber syndrome (Osler-Weber-Rendu disease). Other undesired angiogenesis involves normal processes including ovulation and implantation of a blastula. Accordingly, the compositions described above can be used to block ovulation and implantation of a blastula or to block menstruation (induce amenorrhea).

Another aspect of the invention relates to inhibiting Aurora A activity in a biological sample, which method comprises contacting the biological sample with the Aurora A inhibitor of Formulae I, Ia or Ib, or a composition thereof.

Another aspect of this invention relates to a method of inhibiting Aurora A activity in a patient, which method comprises administering to the patient a compound of Formulae I, la or Ib, or a composition comprising said compound.

In another aspect of this invention, compounds of Formulae I, Ia or Ib are more potent inhibitors of Aurora A compared to Aurora B.

Another aspect of this invention relates to a method of treating or preventing a GSK-3-mediated disease with a GSK-3 inhibitor, which method comprises administering to a patient in need of such a treatment a therapeutically effective amount of a compound of Formulae I, Ia or Ib, or a pharmaceutical composition thereof.

The terms “GSK-3-mediated disease, or “GSK-3-mediated condition”, as used herein, mean any disease or other deleterious condition or state in which GSK-3 is known to play a role. Such diseases or conditions include, without limitation, diabetes, Alzheimer's disease, Huntington's Disease, Parkinson's Disease, AIDS-associated dementia, amyotrophic lateral sclerosis (AML), multiple sclerosis (MS), schizophrenia, cardiomycete hypertrophy, reperfusion/ischemia, and baldness.

One aspect of this invention relates to a method of enhancing glycogen synthesis and/or lowering blood levels of glucose in a patient in need thereof, which method comprises administering to the patient a therapeutically effective amount of a compound of Formulae I, Ia or Ib, or a pharmaceutical composition thereof. This method is especially useful for diabetic patients. Another method relates to inhibiting the production of hyperphosphorylated Tau protein, which is useful in halting or slowing the progression of Alzheimer's disease. Another method relates to inhibiting the phosphorylation of β-catenin, which is useful for treating schizophrenia.

Another aspect of the invention relates to inhibiting GSK-3 activity in a biological sample, which method comprises contacting the biological sample with a GSK-3 inhibitor of Formulae I, Ia or Ib.

Another aspect of this invention relates to a method of inhibiting GSK-3 activity in a patient, which method comprises administering to the patient a compound of Formulae I, Ia or Ib, or a composition comprising said compound.

Another aspect of this invention relates to a method of treating or preventing a CDK-2-mediated disease with a CDK-2 inhibitor, which method comprises administering to a patient in need of such a treatment a therapeutically effective amount of a compound of Formulae I, Ia or Ib, or a pharmaceutical composition thereof.

The terms “CDK-2-mediated disease” or CDK-2-mediated condition”, as used herein, mean any disease or other deleterious condition in which CDK-2 is known to play a role. The terms “CDK-2-mediated disease” or “CDK-2-mediated condition” also mean those diseases or conditions that are alleviated by treatment with a CDK-2 inhibitor. Such conditions include, without limitation, cancer, Alzheimer's disease, restenosis, angiogenesis, glomerulonephritis, cytomegalovirus, HIV, herpes, psoriasis, atherosclerosis, alopecia, and autoimmune diseases such as rheumatoid arthritis, such as are described for example in Fischer, P. M. and Lane, D. P., Current Medicinal Chemistry, 7, 1213-1245 (2000); Mani, S., Wang, C., Wu, K., Francis, R. and Pestell, R., Exp. Opin. Invest. Drugs, 9, 1849 (2000); Fry, D. W. and Garrett, M. D., Current Opinion in Oncologic, Endocrine & Metabolic Investigational Drugs, 2, 40-59 (2000).

Another aspect of the invention relates to inhibiting CDK-2 activity in a biological sample or a patient, which method comprises administering to the patient a compound of Formulae I, Ia or Ib, or a composition comprising said compound.

Another aspect of this invention relates to a method of treating or preventing an ERK-2-mediated diseases with an ERK-2 inhibitor, which method comprises administering to a patient in need of such a treatment a therapeutically effective amount of a compound of Formulae I, Ia or Ib, or a pharmaceutical composition thereof.

The terms “ERK-mediated disease” or “ERK-mediated condition”, as used herein mean any disease or other deleterious condition in which ERK may play a role. The terms “ERK-2-mediated disease” or “ERK-2-mediated condition” also mean those diseases or conditions that are alleviated by treatment with a ERK-2 inhibitor. Such conditions include, without limitation, cancer, stroke, diabetes, hepatomegaly, cardiovascular disease including cardiomegaly, Alzheimer's disease, cystic fibrosis, viral disease, autoimmune diseases, atherosclerosis, restenosis, psoriasis, allergic disorders including asthma, inflammation, neurological disorders and hormone-related diseases. ERK-2 protein kinase and its implication in various diseases has been described for example in Bokemeyer et al. 1996, Kidney Int. 49, 1187; Anderson et al., 1990, Nature 343, 651; Crews et al., 1992, Science 258, 478; Bjorbaek et al., 1995, J. Biol. Chem. 270, 18848; Rouse et al., 1994, Cell 78, 1027; Raingeaud et al., 1996, Mol. Cell Biol. 16, 1247; Raingeaud et al. 1996; Chen et al., 1993 Proc. Natl. Acad. Sci. USA 90, 10952; Oliver et al., 1995, Proc. Soc. Exp. Biol. Med. 210, 162; Moodie et al., 1993, Science 260, 1658; Frey and Mulder, 1997, Cancer Res. 57, 628; Sivaraman et al., 1997, J Clin. Invest. 99, 1478; Whelchel et al., 1997, Am. J. Respir. Cell Mol. Biol. 16, 589.

Another aspect of the invention relates to inhibiting ERK-2 activity in a biological sample or a patient, which method comprises administering to the patient a compound of Formulae I, Ia or Ib, or a composition comprising said compound.

Another aspect of this invention relates to a method of treating or preventing an AKT-mediated diseases with an AKT inhibitor, which method comprises administering to a patient in need of such a treatment a therapeutically effective amount of a compound of Formulae I, Ia or Ib, or a pharmaceutical composition thereof.

The terms “AKT-mediated disease” or “AKT-mediated condition”, as used herein, mean any disease or other deleterious condition in which AKT is known to play a role. The terms “AKT-mediated disease” or “AKT-mediated condition” also mean those diseases or conditions that are alleviated by treatment with a AKT inhibitor. AKT-mediated diseases or conditions include, but are not limited to, proliferative disorders, cancer, and neurodegenerative disorders. The association of AKT, also known as protein kinase B, with various diseases has been described for example in Khwaja, A., Nature, pp. 33-34, 1990; Zang, Q. Y., et al, Oncogene, 19 2000; Kazuhiko, N., et al, The Journal of Neuroscience, 20 2000.

Another aspect of the invention relates to inhibiting AKT activity in a biological sample or a patient, which method comprises administering to the patient a compound of Formulae I, Ia or Ib, or a composition comprising said compound.

Another aspect of this invention relates to a method of treating or preventing a Src-mediated disease with a Src inhibitor, which method comprises administering to a patient in need of such a treatment a therapeutically effective amount of a compound of Formulae I, Ia or Ib, or a pharmaceutical composition thereof.

The terms “Src-mediated disease” or “Src-mediated condition”, as used herein mean any disease or other deleterious condition in which Src is known to play a role. The terms “Src-mediated disease” or “Src-mediated condition” also mean those diseases or conditions that are alleviated by treatment with a Src inhibitor. Such conditions include, without limitation, hypercalcemia, osteoporosis, osteoarthritis, cancer, symptomatic treatment of bone metastasis, and Paget's disease. Src protein kinase and its implication in various diseases has been described for example in Soriano, Cell, 69, 551 (1992); Soriano et al., Cell, 64, 693 (1991); Takayanagi, J. Clin. Invest., 104, 137 (1999); Boschelli, Drugs of the Future 2000, 25(7), 717, (2000); Talamonti, J. Clin. Invest., 91, 53 (1993); Lutz, Biochem. Biophys. Res. 243, 503 (1998); Rosen, J. Biol. Chem., 261, 13754 (1986); Bolen, Proc. Natl. Acad. Sci. USA, 84, 2251 (1987); Masaki, Hepatology, 27, 1257 (1998); Biscardi, Adv. Cancer Res., 76, 61 (1999); Lynch, Leukemia, 7, 1416 (1993); Wiener, Clin. Cancer Res., 5, 2164 (1999); Staley, Cell Growth Diff., 8, 269 (1997).

Another aspect of the invention relates to inhibiting Src activity in a biological sample or a patient, which method comprises administering to the patient a compound of Formulae I, Ia or Ib, or a composition comprising said compound.

Another aspect of this invention relates to a method of treating or preventing an Lck-mediated disease with an Lck inhibitor, which method comprises administering to a patient in need of such a treatment a therapeutically effective amount of a compound of Formulae I, Ia or Ib, or a pharmaceutical composition thereof.

The terms “Lck-mediated disease” or “Lck-mediated condition”, as used herein, mean any disease state or other deleterious condition in which Lck is known to play a role. The terms “Lck-mediated disease” or “Lck-mediated condition” also mean those diseases or conditions that are alleviated by treatment with an Lck inhibitor. Lck-mediated diseases or conditions include, but are not limited to, autoimmune diseases such as transplant rejection, allergies, rheumatoid arthritis, and leukemia. The association of Lck with various diseases has been described for example in Molina et al., Nature, 357, 161 (1992).

Another aspect of the invention relates to inhibiting Lck activity in a biological sample or a patient, which method comprises administering to the patient a compound of Formulae I, Ia or Ib, or a composition comprising said compound.

Another aspect of this invention relates to a method of treating or preventing an Abl-mediated disease with an Abl inhibitor, which method comprises administering to a patient in need of such a treatment a therapeutically effective amount of a compound of Formulae I, Ia or Ib, or a pharmaceutical composition thereof.

The terms “Abl-mediated disease” or “Abl-mediated condition”, as used herein, mean any disease state or other deleterious condition in which Abl is known to play a role. The terms “Abl-mediated disease” or “Abl-mediated condition” also mean those diseases or conditions that are alleviated by treatment with an Abl inhibitor. Abl-mediated diseases or conditions include, but are not limited to, leukemias, particularly chronic myeloid leukemia. The association of Abl with various diseases has been described for example in Druker, et al., N. Engl. J. Med. 2001, 344, 1038-1042.

Another aspect of the invention relates to inhibiting Abl activity in a biological sample or a patient, which method comprises administering to the patient a compound of Formulae I, Ia or Ib, or a composition comprising said compound.

Another aspect of this invention relates to a method of treating or preventing a cKit-mediated disease with an cKit inhibitor, which method comprises administering to a patient in need of such a treatment a therapeutically effective amount of a compound of Formulae I, Ia or Ib, or a pharmaceutical composition thereof.

The terms “cKit-mediated disease” or “cKit-mediated condition”, as used herein, mean any disease state or other deleterious condition in which cKit is known to play a role. The terms “cKit-mediated disease” or “cKit-mediated condition” also mean those diseases or conditions that are alleviated by treatment with an cKit inhibitor. cKit-mediated diseases or conditions include, but are not limited to, mastocytosis/mast cell leukemia, gastrointestinal stromal tumor, sinonasal natural killer/T-cell lymphoma, seminoma/dysgerminoma, throid carcinoma, small-cell lung carcinoma, malignant melanoma, adenoid cystic carcinoma, ovarian carcinoma, acute myelogenious leukemia, anaplastic large-cell lymphoma, angiosarcoma, endometrial carcinom, pediatric T-cell ALL/lymphoma, breast carcinoma and prostate carcinoma. The association of cKit with various diseases has been described for example in Heinrich, et al., J. Clinical Oncology 2002, 20, 1692-1703.

Another aspect of the invention relates to inhibiting cKit activity in a biological sample or a patient, which method comprises administering to the patient a compound of Formulae I, Ia or Ib, or a composition comprising said compound.

Another aspect of this invention relates to a method of treating or preventing a Flt3-mediated disease with an Flt3 inhibitor, which method comprises administering to a patient in need of such a treatment a therapeutically effective amount of a compound of Formulae I, Ia or Ib, or a pharmaceutical composition thereof.

The terms “Flt3-mediated disease” or “Flt3-mediated condition”, as used herein, mean any disease state or other deleterious condition in which Flt3 is known to play a role. The terms “Flt3-mediated disease” or “Flt3-mediated condition” also mean those diseases or conditions that are alleviated by treatment with an Flt3 inhibitor. Flt3-mediated diseases or conditions include, but are not limited to, acute myelogenous leukemia, mixed lineage leukemia and acute lymphocytic leukemia. The association of Flt3 with various diseases has been described for example in Sternberg and Licht, Curr. Opin Hematol. 2004, 12, 7-13.

Another aspect of the invention relates to inhibiting Flt3 activity in a biological sample or a patient, which method comprises administering to the patient a compound of Formulae I, Ia or Ib, or a composition comprising said compound.

Another aspect of this invention relates to a method of treating or preventing a KDR-mediated disease with a KDR inhibitor, which method comprises administering to a patient in need of such a treatment a therapeutically effective amount of a compound of Formulae I, Ia or Ib, or a pharmaceutical composition thereof.

The terms “KDR-mediated disease” or “KDR-mediated condition”, as used herein, mean any disease state or other deleterious condition in which Kinase insert domain-containing receptor (KDR) is known to play a role. The terms “KDR-mediated disease” or “KDR-mediated condition” also mean those diseases or conditions that are alleviated by treatment with an KDR inhibitor. KDR-mediated diseases or conditions include, but are not limited to, carcinoma of the lung, breast, gastrointestinal tract, kidney, bladder, ovary and endometrium, intracranial tumors including glioblatoma multiforme, sporadic capillary hemangioblastoma, hematological malignancies, including T cell lymphoma, acute lymphoblastic leukemia, Burkitt's lymphoma and promyelocytic leukemia, age-related macular degeneration, herpetic ocular disease, rheumatoid arthritis, cerebral ischemia and endometriosis. The association of KDR with various diseases has been described for example in Ferrara, Endocrine Reviews 2004, 25, 581-611.

Another aspect of the invention relates to inhibiting KDR activity in a biological sample or a patient, which method comprises administering to the patient a compound of Formulae I, Ia or Ib, or a composition comprising said compound.

The term “patient” refers to a human, mammal or a veterinary subject.

The term “biological sample”, as used herein, includes, without limitation, cell cultures or extracts thereof; preparations of an enzyme suitable for in vitro assay; biopsied material obtained from a mammal or extracts thereof; and blood, saliva, urine, feces, semen, tears, or other body fluids or extracts thereof.

An amount effective to inhibit protein kinase, for example, Aurora A, is an amount that causes measurable inhibition of the kinase activity when compared to the activity of the enzyme in the absence of an inhibitor. Any method may be used to determine inhibition, such as, for example, the Biological Testing Examples described below.

The term “pharmaceutically acceptable carrier, adjuvant, or vehicle” refers to a non-toxic carrier, adjuvant, or vehicle that may be administered to a patient, together with a compound of this invention, and which does not destroy or reduce the pharmacological activity thereof.

Pharmaceutically acceptable carriers that may be used in these pharmaceutical compositions are generally known in the art. They include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, solvents, salts or electrolytes, such as protamine sulfate, disodium hydrogen phosphate, potassium hydrogen phosphate, sodium chloride, zinc salts, silicates, colloidal silica, magnesium trisilicate, polyvinyl pyrrolidone, cellulose-based substances, polyethylene glycol, sodium carboxymethylcellulose, polyacrylates, waxes, oils, carbohydrate polymers, polyethylene-polyoxypropylene-block polymers, polyethylene glycol and wool fat. Pharmaceutically accepted vehicles can contain mixtures of more than one excipient in which the components and the ratios can be selected to optimize desired characteristics of the formulation including but not limited to shelf-life, stability, drug load, site of delivery, dissolution rate, self-emulsification, control of release rate and site of release, and metabolism.

The compositions of the present invention may be administered orally, intraveneously, subcutaneously, parenterally, by inhalation, topically, rectally, nasally, buccally, vaginally, transdermally, or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, sub-cutaneously, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other surface-active emulsifying agents or bioavailability enhancers which are commonly used in the manufacture of pharmaceutically acceptable solid, liquid, or other dosage forms may also be used for the purposes of formulation.

The pharmaceutical compositions of this invention may be prepared by techniques known in the art and may be orally administered in any orally acceptable dosage form including, but not limited to, capsules, tablets, aqueous suspensions or solutions. In the case of tablets for oral use, carriers commonly used include, but are not limited to, celluloses, lactose, or corn starch. Lubricating agents, such as magnesium stearate, are also typically added. For oral administration in a capsule form, useful diluents or carriers include lactose and dried cornstarch. When aqueous suspensions or solutions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared using techniques known in the art including, for example, by mixing the agent with a suitable non-irritating excipient, which is solid at room temperature but liquid at rectal temperature and, therefore, will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, the airways, or the lower intestinal tract. Suitable topical formulations are readily prepared for each of these areas or organs using techniques known in the art. For example, topical application for the lower intestinal tract can be effected in a rectal suppository formulation (see above) or in a suitable enema formulation. Topically-transdermal patches may also be used.

For topical or transdermal applications, the pharmaceutical compositions may be formulated by techniques known in the art in a suitable ointment or base containing the active component suspended or dissolved in one or more carriers. Carriers for topical administration of the compounds of this invention are well known in the art and include, but are not limited to, mineral oil, liquid petrolatum, white petrolatum, propylene glycol, polyoxyethylene, polyoxypropylene compound, emulsifying wax, and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated by techniques known in the art as micronized or nanometer-sized suspensions in isotonic, pH adjusted sterile saline or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as suspensions or solutions in saline, optionally employing benzyl alcohol or other suitable preservatives, absorption promoters to enhance bioavailability, fluorocarbons, and/or other conventional solubilizing or dispersing agents.

If administered intravenously, carriers can be physiological saline, bacteriostatic water or phosphate buffered saline.

The present invention can be used to treat inflammatory or immune mediated diseases in humans or animals, wherein the inflammatory or immune mediated diseases include, but are not limited to, rheumatoid arthritis, osteoarthritis, ulcerative colitis, Crohn's disease, Mooren's ulcer, arthritis, sarcoidosis, inflammatory or immune mediated bowel disease, systemic lupus, Wegener's syndrome, Stevens-Johnson disease, Behcet's disease, pemphigoid, Lyme's disease, asthma or acquired immune deficiency syndrome.

The present invention can be used to treat infectious diseases in humans or animals, wherein the infectious diseases include, but are not limited to, syphilis, a bacterial infection, a Mycobacterial infection, a bacterial ulcer, a fungal ulcer, a Herpes simplex infection, a Herpes zoster infection, a protozoan infection, malaria, a Bartonellosis infection, or toxoplasmosis.

The present invention can be used to treat blood or blood vessel diseases in humans or animals, wherein the blood or blood vessel diseases include, but are not limited to, vein occlusion, artery occlusion, carotid obstructive disease, polyarteritis, atherosclerosis, Osler-Weber-Rendu disease, sickle cell anemia, leukemia, acute or chronic neoplastic disease of the bone marrow, hemangiomas, hereditary hemorrhagic telangiectasia, disease of the bone marrow, anemia, impaired blood clotting or enlargement of the lymph nodes, liver, or spleen. The present invention can also be used to treat chronic neoplastic disease of the bone marrow, wherein those diseases include, but are not limited to, multiple myeloma and myelo dysplastic syndrome.

The present invention can be used to treat skin conditions in a humans or an animals, wherein the skin conditions include, but are not limited to, abnormal wound healing, acne rosacea, chemical bums of the skin, dermatitis or psoriasis.

In addition, the invention can be used to treat a variety of post-menopausal symptoms, osteoporosis, cardiovascular disease, myocardial angiogenesis, plaque neovascularization, hemophiliac joints, angiofibroma, wound granulation, intestinal adhesions, scleroderma, hypertrophic scars; i.e., keloids. They are also useful in the treatment of diseases that have angiogenesis as a pathologic consequence, such as cat scratch disease, and Helicobacter pylori ulcers. The invention can also be used to treat Alzheimer's disease, to reduce the incidence of stroke, and as an alternative to prior estrogen replacement therapies. The compounds of the present invention can work by estrogenic and non-estrogenic biochemical pathways.

Additionally, the compounds of the present invention can be used to treat endometriosis. Endometriosis is the abnormal growth of endometrial cells; the same cells that line the uterus that are shed monthly in the menstrual process. Wayward endometrial cells can position themselves in the lower abdomen on areas such as the cul-de-sac, the recto-vaginal septum, the stomach, the fallopian tubes, the ovaries, and the bladder. During menstruation, the normal uterine lining is sloughed off and expelled through the vagina, but transplanted endometrial tissue has no means of exiting the body; instead the endometrial tissue and cells adhere and grow where positioned. The results are internal bleeding, inflammation, and scarring. One of the serious consequences of endometrial scarring is infertility. The endometrial growths are generally not malignant or cancerous. Among other complications, the growths can rupture and can spread the endometriosis to new areas of the lower abdomen. Endometriosis is a progressive disease. The growths or lesions are first seen as clear vesicles, then become red, and finally progress to black lesions over a period of seven to ten years.

In addition, the compounds of this invention, can be formulated to increase the bioavailability of the compound by methods well known to those of ordinary skill in the art. Methods of formulating the compounds of this invention and examples of formulations are described in “Water-Insoluble Drug Formulation” Rong Liu editor, CRC Press LLC, 2000, which is incorporated herein by reference in its entirety.

Formulations contemplated as part of this invention include, but are not limited to, nanoparticles formulations made by controlled precipitation methods and by methods disclosed in U.S. patent application Ser. No. 10/392,403 (Publication No. 2004/0033267), which is hereby incorporated by reference in its entirety. Common excipients for nanoparticles known in the art include water, surface active agents such as sugar polymers (modified celluloses) and detergents, and also optionally preservatives such as benzalkonium salts, benzoic acid or salts thereof, or parabens. By forming nanoparticles, the compositions disclosed herein have increased bioavailability. Preferably, the particles of the compounds of the present invention have an effective average particle size of less than about 2 microns, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods well known to those of ordinary skill in the art. Nanoparticle preparations can be incorporated into many of the formulation approaches described here, including for example suspensions or creams or ointments for topical or transdermal administration, suspensions or powders or tablets or capsules or pellets for suppositories or for oral administration, suspensions for sterile injectable formulations, and polymer formulations.

In one embodiment, the active compounds are prepared with carriers that will protect the compound against rapid elimination from the body, such as a controlled release formulation, including implants and microencapsulated delivery systems. The compounds can be incorporated into biodegradable or non-biodegradable polymers allowing for sustained release of the compound. The polymers can be implanted so that the drug is delivered parenterally throughout the body or the polymers with the compounds that make up this invention can be implanted in the vicinity of the tumor. A review of polymers in controlled drug delivery can be found for example in “Biodegradable Polymers as Drug Delivery Systems, Chasin M and Langer R (eds), New York, Marcel Dekker, 1990, which is incorporated herein by reference in its entirety. Another review can be found in “Handbook of Biodegradable Polymers”, D. Weseman, J. Kost and A. Domb, Taylor & Francis, 1998, which is incorporated herein by reference in its entirety. Biodegradable, biocompatible polymers such as ethylene vinyl acetate, polyanhydrides, polyglycolic acid, collagen, polyorthoesters, and polylactic acid, can be used. Methods for preparation of such formulations will be apparent to those skilled in the art. Liposomal suspensions (including liposomes targeted to infected cells with monoclonal antibodies to viral antigens) are also preferred as pharmaceutically acceptable carriers. These may be prepared according to methods known to those skilled in the art. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the compound is then introduced into the container. The container is then swirled by hand to free lipid material from the sides of the container and to disperse lipid aggregates, thereby forming the liposomal suspension.

A “pharmaceutically acceptable derivative or prodrug” means any pharmaceutically acceptable salt, ester, amide, salt of an ester or amide, or other derivative of a compound of this invention which, upon administration to a recipient, is capable of providing, either directly or indirectly, a compound of this invention or an inhibitorily active metabolite or residue thereof. Particularly favored derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Pharmaceutically acceptable prodrugs of the compounds of this invention include, without limitation, the following derivatives of the present compounds: esters, amino acid esters, amino acid amides, phosphate esters, metal salts, sulfonate esters, carbamates, and amides.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N⁺(C₁₋₄ alkyl)₄ salts. This invention also envisions the quaternization of any basic nitrogen-containing groups of the compounds disclosed herein. Water or oil-soluble or dispersible products may be obtained by such quaternization.

Compounds of this invention can also be formulated as mixtures or complexes, including, but not limited to, host-guest complexes with molecules such as cyclodextrins, non-ionic complexes, stabilized amorphous solids, glasses, solid solutions, and co-precipitates. The compound in these formulations can be dispersed to individual molecules, amorphous particles, or crystalline particles. These formulations can be prepared by techniques known to those skilled in the art including, but not limited to, solvent-mediated co-precipitation, spray-drying, grinding, hot-melt extrusion, and granulation.

The amount of the protein kinase inhibitor that may be combined with the carrier materials to produce a single dosage form will vary depending upon the patient treated and the particular mode of administration. Preferably, the compositions should be formulated so that a dosage of between 0.01-100 mg/kg body weight/day of the inhibitor can be administered to a patient receiving these compositions. The compound is conveniently administered in any suitable dosage form including, but not limited to, one containing 7-3000 mg or 70-1400 mg of active ingredient per unit dosage form. An oral dosage of 50-1000 mg is usually convenient.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of the inhibitor will also depend upon the particular compound in the composition.

Depending upon the particular protein kinase-mediated condition to be treated or prevented, additional therapeutic agents, which are normally administered to treat or prevent that condition, may be administered together with the inhibitors of this invention. For example, in the treatment of cancer, other kinase inhibitors, chemotherapeutic agents, anti-angiogenesis agents, anti-nausea agents, colony-stimulating factors, or other anti-proliferative agents may be combined with the present compounds to treat cancer as is known in the art. These agents include, without limitation, bevacizumab, adriamycin, dexamethasone, vincristine, cyclophosphamide, fluorouracil, topotecan, taxanes, interferons, and platinum derivatives.

Other examples of agents the inhibitors of this invention may also be combined with include, without limitation, agents for treating diabetes such as insulin or insulin analogues, in injectable or inhalation form, glitazones, alpha glucosidase inhibitors, biguanides, insulin sensitizers, and sulfonyl ureas; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; therapeutic antibodies such as bevacizumab; and agents for treating immunodeficiency disorders such as gamma globulin.

Those additional agents may be administered separately from the protein kinase inhibitor-containing composition, or as part of a multiple dosage regimen. Alternatively, those agents may be part of a single dosage form, mixed together with the protein kinase inhibitor of this invention in a single composition.

Compounds of this invention may exist in alternative tautomeric forms, for example as in tautomers shown below. Unless otherwise indicated, the representation of any tautomer is meant to include any other tautomers.

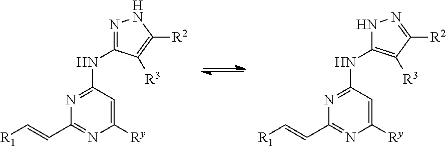

In one embodiment, the present invention provides a compound of Formula I or a pharmaceutically acceptable derivative or prodrug thereof,

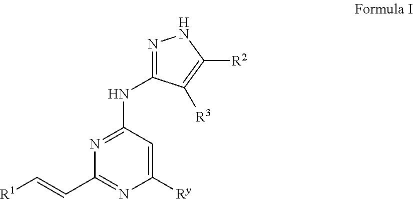

wherein:

R^{y} is

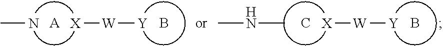

X is N, C or CR;

Y is N, C, or CR;

Ring A is an optionally substituted 4, 5 or 6 membered monocyclic heterocyclic ring;

Ring B is an optionally substituted 3-7 membered monocyclic or 8-10 membered bicyclic ring selected from carbocyclyl, aryl, heterocyclyl, or heteroaryl;

Ring C is an optionally substituted 3, 4, 5 or 6 membered heterocyclic, carbocyclic, aryl or heteroaryl ring;

W is (CR₂)ₙ, where n is 0, 1, 2, 3, 4 or 5;

R¹ is an optionally substituted monocyclic or bicyclic aryl ring;

R² and R³ are independently hydrogen, optionally substituted aliphatic, optionally substituted carbocyclyl, optionally substituted aryl, optionally substituted heteroaryl, optionally substituted heterocyclyl, or R² and R³ taken together with their intervening atoms form a fused, optionally substituted unsaturated or partially unsaturated ring having 0-3 ring heteroatoms; and

R is hydrogen, aliphatic, aryl, aralkyl, alkylaryl, heterocyclyl, heteroaryl, halo, hydroxyl, alkoxy, amino, alkylamino, or dialkylamino.

In one embodiment of Formula I, R^{y} is

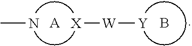

In another embodiment of Formula I, R^{y} is

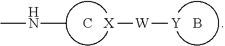

In another embodiment, a compound of Formula I is provided where R² is hydrogen or optionally substituted aliphatic. In another embodiment, R² is methyl.

In another embodiment of Formula I, R¹ is phenyl.

In one embodiment, R³ is hydrogen.

In another embodiment of Formula I, R² and R³ are independently hydrogen, optionally substituted aliphatic, optionally substituted carbocyclyl, optionally substituted aryl, optionally substituted heteroaryl, optionally substituted heterocyclyl, and R¹ is optionally substituted phenyl.

In still another embodiment, R¹ is optionally substituted phenyl, R² and R³ are independently hydrogen, optionally substituted aliphatic, optionally substituted carbocyclyl, optionally substituted aryl, optionally substituted heteroaryl, optionally substituted heterocyclyl, R^{y} is

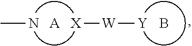

Ring A is a 4, 5 or 6 membered monocyclic heterocyclic ring; and Ring B is a 3-7 membered monocyclic carbocyclyl, aryl, heterocyclyl, or heteroaryl ring.

In yet another embodiment of Formula I, R¹ is optionally substituted phenyl, R² and R³ are independently hydrogen, optionally substituted aliphatic, optionally substituted carbocyclyl, optionally substituted aryl, optionally substituted heteroaryl, optionally substituted heterocyclyl and R^{y} is

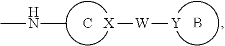

Ring C is a 4, 5 or 6 membered heterocyclic, carbocyclic, aryl or heteroaryl ring; and Ring B is a 3-7 membered monocyclic carbocyclyl, aryl, heterocyclyl, or heteroaryl ring.

In some embodiments of Formula I, Ring A is a substituted 4-membered heterocyclic ring. In other embodiments of Formula I, Ring A is a 5 or 6 membered heterocyclic ring.

In other embodiments, Ring B is an optionally substituted aryl or heteroaryl ring. In other embodiments, Ring B is an optionally substituted carbocyclic or heterocyclic ring. In other embodiments, Ring B is not aryl, for example substituted or unsubstituted phenyl.

In still another embodiment of Formula I, Ring C is an optionally substituted aryl ring or an optionally substituted carbocyclic ring.

In another embodiment of Formula I, Ring C is an optionally substituted heteroaryl or heterocyclic ring.

In another embodiment of Formula I, R² is aliphatic, R³ is H, R¹ is optionally substituted phenyl, and Ring A is a 4 membered heterocyclic ring.

In still another embodiment of Formula I, R² is aliphatic, R³ is H, R¹ is optionally substituted phenyl, and Ring A is a 5 or 6 membered heterocyclic ring.

In other embodiments of Formula I, R¹ is optionally substituted phenyl, R² is aliphatic, R³ is H and Ring B is a an optionally substituted heterocyclyl or heteroaryl ring.

In yet another embodiment of Formula I, R¹ is optionally substituted phenyl, R² is aliphatic, R³ is H and Ring B is an optionally substituted aryl or carbocyclic ring.

In some embodiments, R¹ is optionally substituted phenyl, R² is aliphatic, R³ is H, Ring A is a 4 membered ring, and Ring B is an optionally substituted carbocyclic or aryl ring.

In other embodiments, R¹ is optionally substituted phenyl, R² is aliphatic, R³ is H, Ring A is a 4 membered ring, and Ring B is an optionally substituted heterocyclic or heteroaryl ring.

In yet another embodiment of the invention, R¹ is optionally substituted phenyl, R² is aliphatic, R³ is H, Ring C is an optionally substituted aryl or heterocyclic ring, and Ring B is an optionally substituted heterocyclic or heteroaryl ring.

In some embodiments, W is (CR₂)ₙ where n is 1 or 2. In other embodiments, n is 0.

In other embodiments, R¹ is optionally substituted phenyl, R² is aliphatic, R³ is H, and Ring A is a substituted azetidine ring.

In other embodiments of Formula I, R¹ is optionally substituted phenyl, R² is aliphatic, R³ is H, and Ring A is an optionally substituted pyrrolidine, piperidine or piperazine ring.

In another embodiment of Formula I, R¹ is optionally substituted phenyl, R² is aliphatic, R³ is H, and Ring B is an optionally substituted pyrrolidine, piperidine, morpholine, piperazine, tetrahydrofuran, tetrahydropyran, phenyl, pyridine, furan, indole, pyrimidine or homopiperidine ring.

In another embodiment of Formula I, R¹ is optionally substituted phenyl, R² is aliphatic, R³ is H, and Ring C is an optionally substituted phenyl, pyrrolidine, piperidine or piperazine ring.

In another embodiment of the invention, a compound of Formula I is provided wherein R¹ is optionally substituted phenyl, R² is methyl, R³ is H or aliphatic, Ring A is an optionally substituted azetidine ring, and Ring B is an optionally substituted carbocyclic, aryl, heterocyclic or heteroaryl ring.

In another embodiment of the invention, a compound of Formula I is provided wherein R¹ is optionally substituted phenyl, R² is methyl, R³ is H or aliphatic, and Ring A is an optionally substituted pyrrolidine, piperidine or piperazine ring, and Ring B is an optionally substituted carbocyclic, aryl, heterocyclic or heteroaryl ring.

In another embodiment of the invention, a compound of Formula I is provided wherein R¹ is optionally substituted phenyl, R² is methyl, R³ is H or aliphatic, and Ring C is an optionally substituted pyrrolidine, piperidine or piperazine ring, and Ring B is an optionally substituted carbocyclic, aryl, heterocyclic or heteroaryl ring.

In yet another embodiment, a compound of Formula I is provided wherein R¹ is optionally substituted phenyl, R² is methyl, R³ is H or aliphatic, and Ring A is an optionally substituted pyrrolidine, piperidine or piperazine ring, and Ring B is an optionally substituted pyrrolidine, piperidine, morpholine, piperazine, tetrahydrofuran, tetrahydropyran, phenyl, pyridine, furan, indole, pyrimidine or homopiperidine ring.

Another embodiment provides a compound of Formula I, wherein R¹ is a substituted phenyl group substituted by one or more of alkyl, alkoxy, halogen, CF₃, amino, alkylamino, dialkylamino, cyano and nitro.

In another aspect of the invention a compound of the Formula Ia or a pharmaceutically acceptable derivative or prodrug thereof is provided:

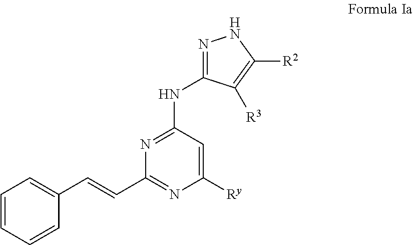

wherein R^{y}, R², R³, and n are as defined for Formula I above.

In one embodiment of Formula Ia, R^{y} is

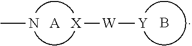

In another embodiment of Formula Ia, R^{y} is

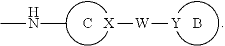

In another embodiment, a compound of Formula Ia is provided where R² is hydrogen or optionally substituted aliphatic. In another embodiment, R² is methyl, and R³ is H.

In still another embodiment, R² and R³ are independently hydrogen, optionally substituted aliphatic, optionally substituted carbocyclyl, optionally substituted aryl, optionally substituted heteroaryl, optionally substituted heterocyclyl, and R^{y} is

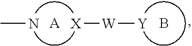

Ring A is a 4, 5 or 6 membered monocyclic heterocyclic ring; and Ring B is a 3-7 membered monocyclic carbocyclyl, aryl, heterocyclyl, or heteroaryl ring.

In yet another embodiment of Formula Ia, R² and R³ are independently hydrogen, optionally substituted aliphatic, optionally substituted carbocyclyl, optionally substituted aryl, optionally substituted heteroaryl, optionally substituted heterocyclyl and R^{y} is

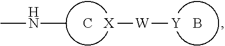

Ring C is a 4, 5 or 6 membered heterocyclic, carbocyclic, aryl or heteroaryl ring; and Ring B is a 3-7 membered monocyclic carbocyclyl, aryl, heterocyclyl, or heteroaryl ring.

In some embodiments of Formula Ia, Ring A is a substituted 4-membered heterocyclic ring. In other embodiments of Formula Ia, Ring A is a 5 or 6 membered heterocyclic ring.

In other embodiments, Ring B is an optionally substituted aryl or heteroaryl ring. In other embodiments, Ring B is an optionally substituted carbocyclic or heterocyclic ring.

In still another embodiment of Formula Ia, Ring C is an optionally substituted aryl ring or an optionally substituted carbocyclic ring.

In another embodiment of Formula Ia, Ring C is an optionally substituted heteroaryl or heterocyclic ring.

In another embodiment of Formula Ia, R² is aliphatic, R³ is H, and Ring A is a 4 membered heterocyclic ring.

In still another embodiment of Formula Ia, R² is aliphatic, R³ is H, and Ring A is a 5 or 6 membered heterocyclic ring.

In other embodiments of Formula Ia, R² is aliphatic, R³ is H, and Ring B is a an optionally substituted heterocyclyl or heteroaryl ring.

In yet another embodiment of Formula Ia, R² is aliphatic, R³ is H, and Ring B is an optionally substituted aryl or carbocyclic ring.

In some embodiments of Formula Ia, R² is aliphatic, R³ is H, Ring A is a 4 membered ring, and Ring B is an optionally substituted carbocyclic or aryl ring.

In other embodiments, R² is aliphatic, R³ is H, Ring A is a 4 membered ring, and Ring B is an optionally substituted heterocyclic or heteroaryl ring.

In yet another embodiment of Formula Ia, R² is aliphatic, R³ is H, Ring C is an optionally substituted aryl or heterocyclic ring, and Ring B is an optionally substituted heterocyclic or heteroaryl ring.

In some embodiments of Formula Ia, W is (CR₂)ₙ where n is 1 or 2. In other embodiments, n is 0.

In other embodiments, R² is aliphatic, R³ is H, and Ring A is an optionally substituted azetidine ring.

In other embodiments of Formula Ia, R² is aliphatic, R³ is H, and Ring A is an optionally substituted pyrrolidine, piperidine or piperazine ring.

In another embodiment of Formula Ia, R² is aliphatic, R³ is H, and Ring B is an optionally substituted pyrrolidine, piperidine, morpholine, piperazine, tetrahydrofuran, tetrahydropyran, phenyl, pyridine, furan, indole, pyrimidine or homopiperidine ring.

In another embodiment of Formula Ia, R² is aliphatic, R³ is H, and Ring C is an optionally substituted phenyl, pyrrolidine, piperidine or piperazine ring.

In another embodiment of the invention, a compound of Formula Ia is provided wherein R² is methyl, R³ is H or aliphatic, Ring A is an optionally substituted azetidine ring, and Ring B is an optionally substituted carbocyclic, aryl, heterocyclic or heteroaryl ring.

In another embodiment of the invention, a compound of Formula Ia is provided wherein R² is methyl, R³ is H or aliphatic, and Ring A is an optionally substituted pyrrolidine, piperidine or piperazine ring, and Ring B is an optionally substituted carbocyclic, aryl, heterocyclic or heteroaryl ring.

In another embodiment of the invention, a compound of Formula Ia is provided wherein R² is methyl, R³ is H or aliphatic, and Ring C is an optionally substituted pyrrolidine, piperidine or piperazine ring, and Ring B is an optionally substituted carbocyclic, aryl, heterocyclic or heteroaryl ring.

In yet another embodiment, a compound of Formula Ia is provided wherein R² is methyl, R³ is H or aliphatic, and Ring A is an optionally substituted pyrrolidine, piperidine or piperazine ring, and Ring B is an optionally substituted pyrrolidine, piperidine, morpholine, piperazine, tetrahydrofuran, tetrahydropyran, phenyl, pyridine, furan, indole, pyrimidine or homopiperidine ring.

In another embodiment a compound of the Formula Ib or a pharmaceutically acceptable derivative or prodrug thereof is provided:

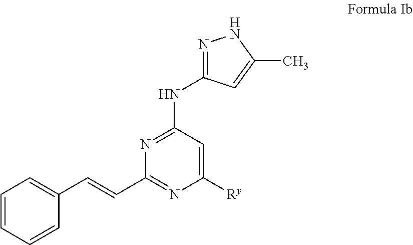

wherein R^{y} is as defined for Formula I above.

In one embodiment of Formula Ib, R^{y} is

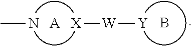

In another embodiment of Formula Ib, R^{y} is

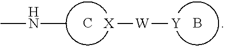

In some embodiments of Formula Ib, Ring A is a substituted 4-membered heterocyclic ring. In other embodiments of Formula Ib, Ring A is a 5 or 6 membered heterocyclic ring.

In other embodiments, Ring B is an optionally substituted aryl or heteroaryl ring. In other embodiments, Ring B is an optionally substituted carbocyclic or heterocyclic ring.

In still another embodiment of Formula Ib, Ring C is an optionally substituted aryl or heterocyclic ring.

In another embodiment of Formula Ib, Ring A is an optionally substituted azetidine ring. In another embodiment of Formula Ib, Ring A is an optionally substituted pyrrolidine, piperidine, or piperazine ring.

In one embodiment of Formula Ib, Ring B is an optionally substituted pyrrolidine, piperidine, morpholine, phenyl, pyridine, pyrimidine, tetrahydrofuran, tetrahydropyran or homopiperidine ring.

In another embodiment, Ring C is an optionally substituted pyrrolidine, piperidine or phenyl ring.

In some embodiments, W is (CR₂)ₙ where n is 1 or 2. In other embodiments, n is 0.

In another embodiment of the invention, a compound of Formula Ib is provided wherein Ring A is an optionally substituted azetidine ring, and Ring B is an optionally substituted carbocyclic, pyrrolidine, piperidine, morpholine, phenyl, pyridine, pyrimidine, tetrahydrofuran, tetrahydropyran or a homopiperidine ring.

In another embodiment of the invention, a compound of Formula Ib is provided wherein Ring A is an optionally substituted pyrrolidine, piperidine or piperazine ring, and Ring B is an optionally substituted carbocyclic, pyrrolidine, piperidine, morpholine, phenyl, pyridine, pyrimidine, tetrahydrofuran, tetrahydropyran or a homopiperidine ring.

In still another embodiment, a compound of Formula Ib is provided, wherein Ring A is an optionally substituted piperidine ring and Ring B is an optionally substituted piperidine, phenyl, morpholine, pyridine, or pyrimidine ring.

In another embodiment of the invention, a compound of Formula Ib is provided wherein Ring C is an optionally substituted pyrrolidine, piperidine or piperazine ring, and Ring B is an optionally substituted piperidine, phenyl, morpholine, pyridine, or pyrimidine ring.

In another embodiment of the invention, a compound of Formula Ib is provided wherein Ring C is an optionally substituted pyrrolidine, piperidine or piperazine ring, and Ring B is an optionally substituted piperidine, morpholine, pyridine, or pyrimidine ring.

In another embodiment, Ring A is an optionally substituted piperazine ring, and Ring B is an optionally substituted piperidine, phenyl, morpholine, pyridine, or pyrimidine ring.

In still another embodiment, Ring A is an optionally piperidine ring or an optionally substituted piperazine ring, Ring B is an optionally substituted morpholine, phenyl, tetrahydrofuran, piperidine or homopiperidine ring, W is (CH₂)ₙ, and n is 1 or 2.

In another embodiment of Formula Ib, Ring B is an optionally substituted azetidinyl ring or an optionally substituted piperidine ring, where X is CR and R is OH.

In one embodiment, Ring B is a phenyl ring substituted by 1, 2 or 3 halogen atoms, wherein the halogen atoms may be the same or different.

In another embodiment of Formula Ib, Ring B is a phenyl ring substituted with one or more substituents selected from alkoxy, cyano, nitro, amino, alkylamino or dialkylamino.

In one embodiment, the compound is a compound of Formula I, or a pharmaceutically acceptable derivative or prodrug thereof, wherein:

R^{y} is

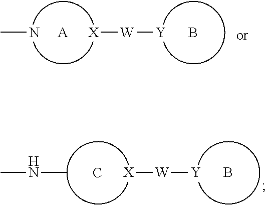

X is N, C or CR;

Y is N, C, or CR;

Ring A is an optionally substituted 4, 5 or 6 membered monocyclic heterocyclic ring;

Ring B is an optionally substituted 3-7 membered monocyclic or 8-10 membered bicyclic ring selected from carbocyclyl, aryl, heterocyclyl, or heteroaryl;

Ring C is an optionally substituted 3, 4, 5 or 6 membered heterocyclic, carbocyclic, aryl or heteroaryl ring;

W is (CR₂)ₙ, where n is 0, 1, 2, 3, 4 or 5;

R¹ is an optionally substituted monocyclic or bicyclic aryl ring;

R² and R³ are independently hydrogen, optionally substituted aliphatic, optionally substituted carbocyclyl, optionally substituted aryl, optionally substituted heteroaryl, optionally substituted heterocyclyl, or R² and R³ taken together with their intervening atoms form a fused, optionally substituted unsaturated or partially unsaturated ring having 0-3 ring heteroatoms; and

R is hydrogen, aliphatic, aryl, aralkyl, alkylaryl, heterocyclyl, heteroaryl, halo, hydroxyl, alkoxy, amino, alkylamino, or dialkylamino.

In another embodiment, the compound is a compound of Formula I, or a pharmaceutically acceptable derivative or prodrug thereof, wherein:

R^{y} is

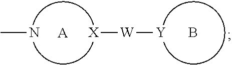

X is N, C or CR;

Y is N, C, or CR;

Ring A is an optionally substituted 4, 5 or 6 membered monocyclic heterocyclic ring;

Ring B is an optionally substituted 3-7 membered monocyclic or 8-10 membered bicyclic ring selected from carbocyclyl, aryl, heterocyclyl, or heteroaryl;

W is (CR₂)ₙ, where n is 0, 1, 2, 3, 4 or 5;

R¹ is an optionally substituted monocyclic or bicyclic aryl ring;

R² and R³ are independently hydrogen, optionally substituted aliphatic, optionally substituted carbocyclyl, optionally substituted aryl, optionally substituted heteroaryl, optionally substituted heterocyclyl; and

R is hydrogen, aliphatic, aryl, aralkyl, alkylaryl, heterocyclyl, heteroaryl, halo, hydroxyl, alkoxy, amino, alkylamino, or dialkylamino.

In one embodiment of Formulae I, Ia, or Ib, Ring A is 4, 5 or 6 membered monocyclic heterocyclyl or heteroaryl. In a subembodiment, Ring A is heterocyclyl. For example, Ring A is selected from the group consisting of azetidinyl, piperidinyl, piperazinyl, and pyrrolidinyl, all of which may be substituted or unsubstituted. In certain embodiments, Ring A is substituted, for example hydroxy-substituted-piperidinyl.

In one embodiment of Formulae I, Ia, or Ib, Ring C is aryl, for example phenyl.

In one embodiment of Formulae I, Ia, or Ib, Ring B is 3, 4, 5, 6 or 7 membered monocyclic carbocyclyl, aryl, heterocyclyl, or heteroaryl ring. For example, Ring B is selected from the group consisting of cyclopropyl, cyclohexyl, phenyl, piperidinyl, piperazinyl, pyrrolidinyl, morpholinyl, pyridinyl, diazinyl, tetrahydrofuranyl, and azepanyl, all of which may be substituted or unsubstituted. In certain embodiments, Ring B is substituted, for example hydroxy-substituted-piperidinyl or hal-substituted phenyl. In certain embodiments, Ring B is carbocyclyl, for example cyclopropyl, cyclobutyl, cyclopentyl, cyclohexyl, or cycloheptyl. In certain embodiments, Ring B is phenyl. In other embodiments, Ring B is heterocyclyl or heteroaryl, for example, aziridinyl, azirinyl, oxiranyl, oxirenyl, thiiranyl, thiirenyl, dioxiranyl, diazirinyl, azetidinyl, azetyl, oxetanyl, oxetyl, thietanyl, thietyl, diazetidinyl, doxetanyl, dioxetenyl, dithietanyl, dithetyl, pyrrolidinyl, pyrrolinyl, pyrrolyl, tetrahydrofuranyl, dihydrofuranyl, furanyl, tetrahydrothiophenyl, dihydrothiophenyl, thiophenyl, imidazolidinyl, pyrazolidinyl, imidazolyl, imidazolinyl, pyrazolyl, pyrazolinyl, oxazolidinyl, isoxazolidinyl, oxazolyl, oxazolinyl, isoxazolyl, isoxazolinyl, dioxolanyl, oxathiolanyl, dithiolanyl, triazolyl, dithiazolyl, furazanyl, oxadiazolyl, thiadiazolyl, tetrazolyl, piperidinyl, pyridinyl, tetrahydropyranyl, pyranyl, thianyl, thiopyranyl, piperazinyl, diazinyl, morpholinyl, oxazinyl, thiazinyl, dithianyl, dioxanyl, dioxinyl, triazinyl, trioxanyl, tetrazinyl, azepanyl, homopiperidinyl, azepinyl, oxepanyl, pxepinyl, thiepanyl, thiepinyl, diazepinyl, or thiazepinyl, all of which may be substituted or unsubstituted.

In one embodiment of Formula I, Ia or Ib, Ring A is selected from the group consisting of azetidinyl, piperidinyl, piperazinyl, and pyrrolidinyl, all of which may be substituted or unsubstituted, and Ring B is selected from the group consisting of cyclopropyl, cyclohexyl, phenyl, piperidinyl, piperazinyl, pyrrolidinyl, morpholinyl, pyridinyl, diazinyl, tetrahydrofuranyl, and azepanyl, all of which may be substituted or unsubstituted.

In one embodiment of Formula I, Ia, or Ib, Ry is

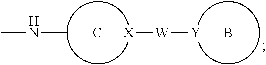

Ring C is aryl, for example phenyl, and Ring B is heterocyclyl, for example azepanyl.

In one embodiment of Formulae I, Ia or Ib, the optionally substituted or fused-ring amino-pyrazole can for example be selected from the following structures:

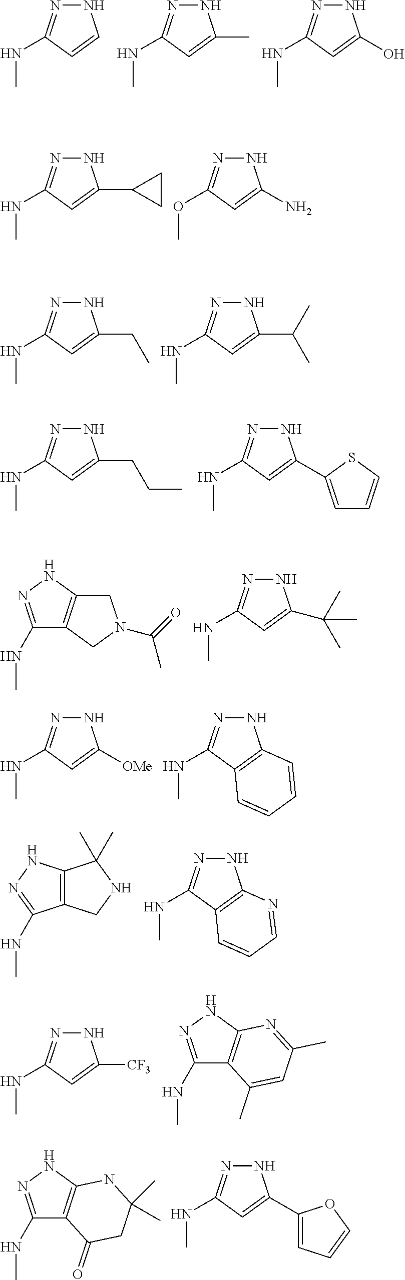

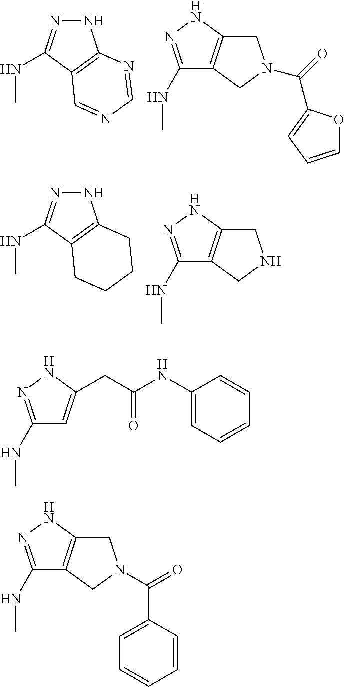

R² and R³ may be taken together to form a fused ring, thus providing a bicyclic ring system containing a pyrazole ring. Fused rings include benzo, pyrido, pyrimido, a partially unsaturated 6-membered carbocyclo ring, wherein said fused ring is optionally substituted. Fused 5-membered rings are also envisioned and include, but are not limited to, pyrrolo, tetrahydrofuran, tetrahydrothiofuran imidazolidine and pyrazolidine. These are exemplified in the following Formula I compounds having a pyrazole-containing bicyclic ring system:

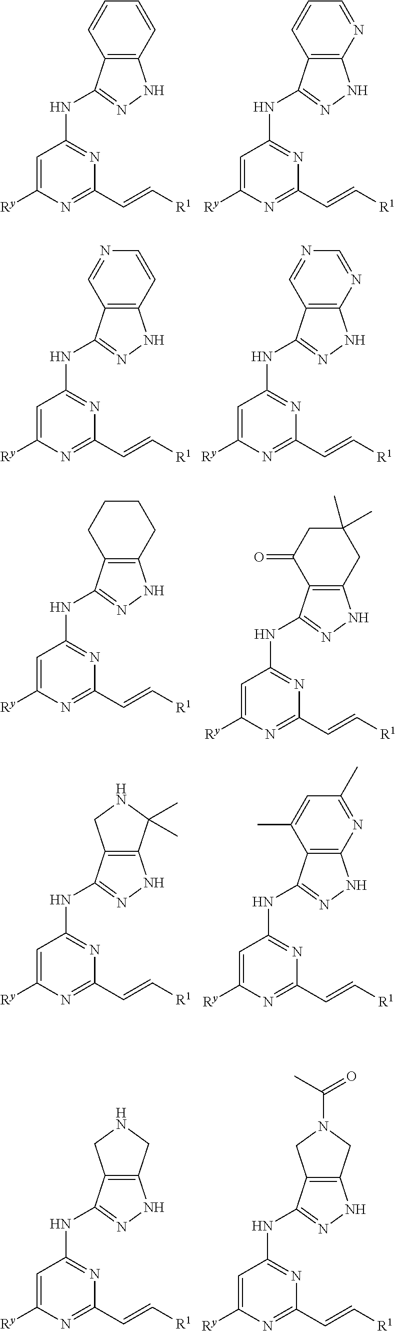

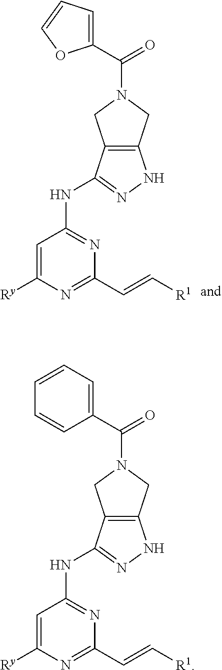

Substituents on the R²/R³ fused rings include one or more of the following: -halo, —N(R⁴)₂, —C₁₋₃ alkyl, —C₁₋₃ haloalkyl, —NO₂, —O(C₁₋₃ alkyl), —CO₂(C₁₋₃ alkyl), —CN, —SO₂(C₁₋₃ alkyl), —SO₂NH₂, —OC(O)NH₂, —NH₂SO₂(C₁₋₃ alkyl), —NHC(O)(C₁₋₃ alkyl), —C(O)NH₂, and —CO(C₁₋₃ alkyl), in one embodiment, the C₁₋₃ alkyl is methyl.

When R¹ of Formula I is bicyclic, optionally substituted bicyclic R¹ groups include naphthyl and anthracenyl.

In some embodiments, the invention provides compounds of Formula I wherein R¹ is selected from the following group:

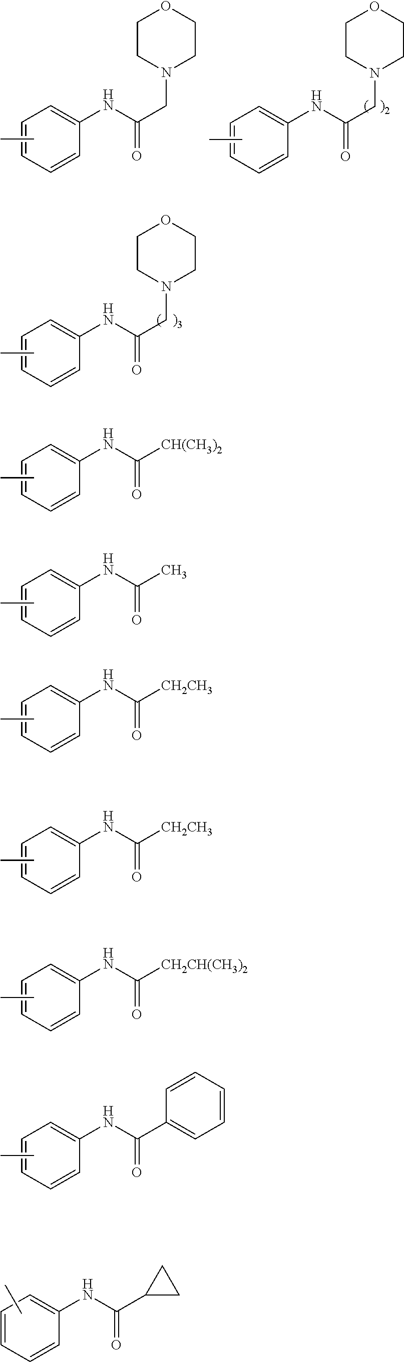

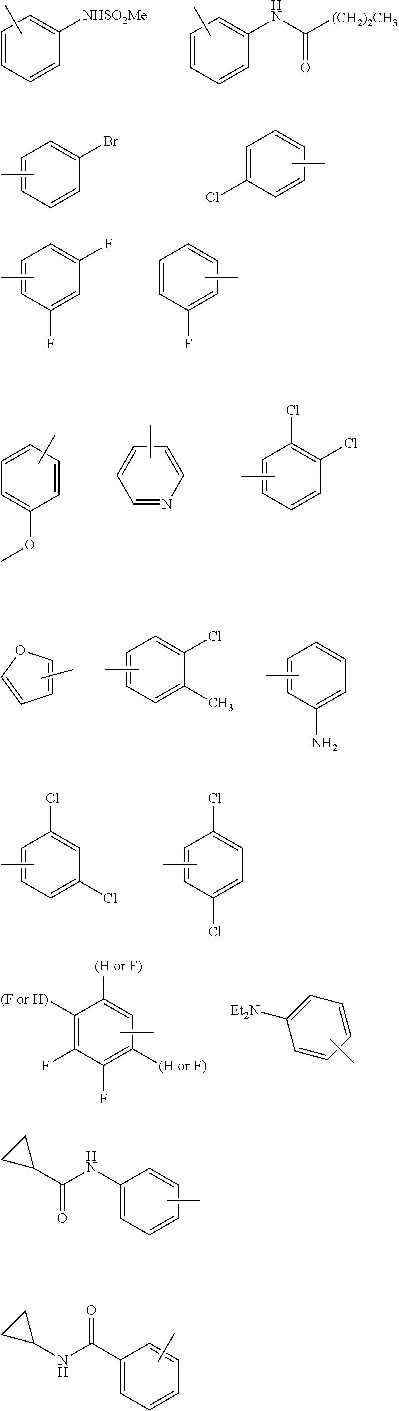

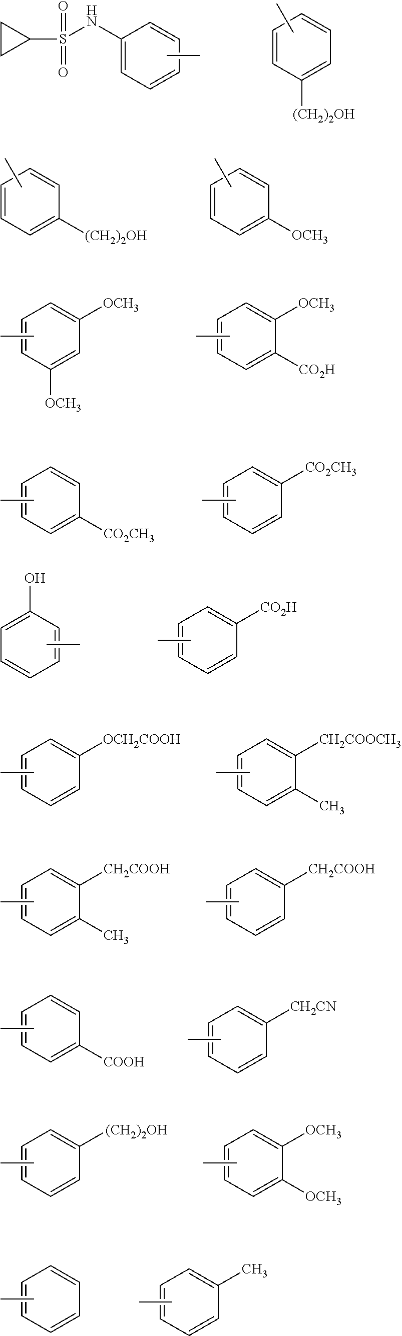

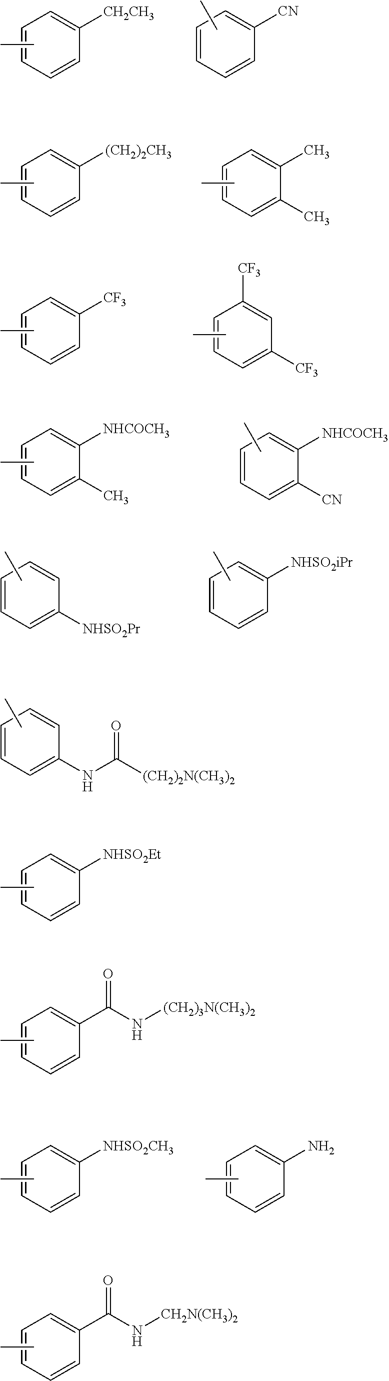

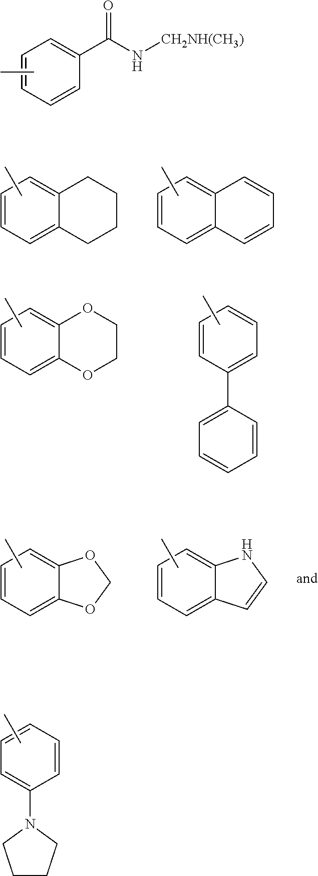

where the line drawn through the side of the substituent indicates that the substituent can be joined to the pyrimidine ring at any substitutable ring atom.

In some embodiments of Formulae I, Ia or Ib, R^{y} is selected from:

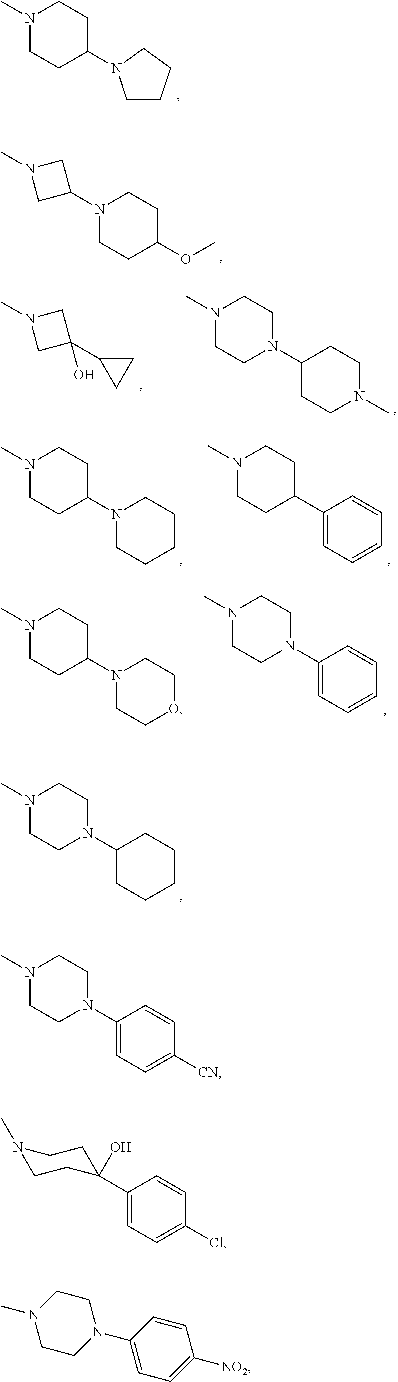

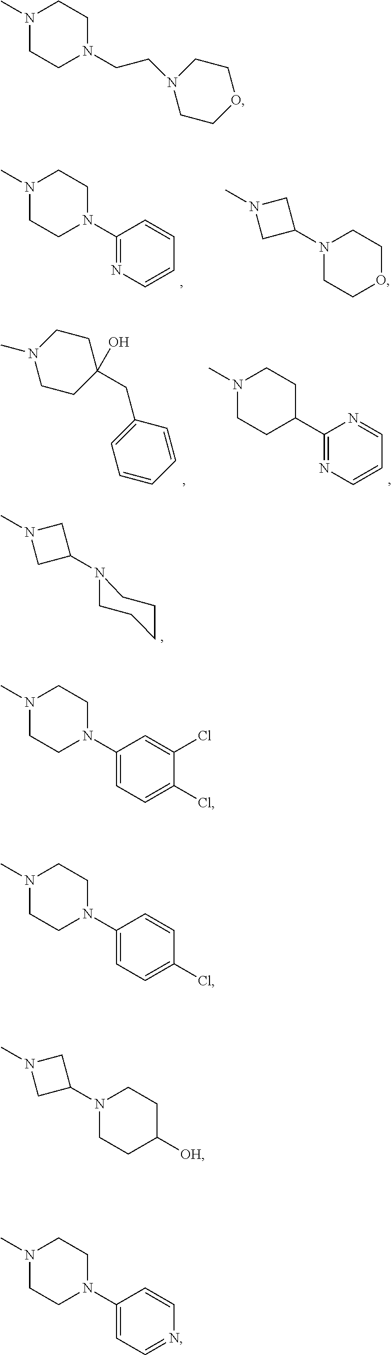

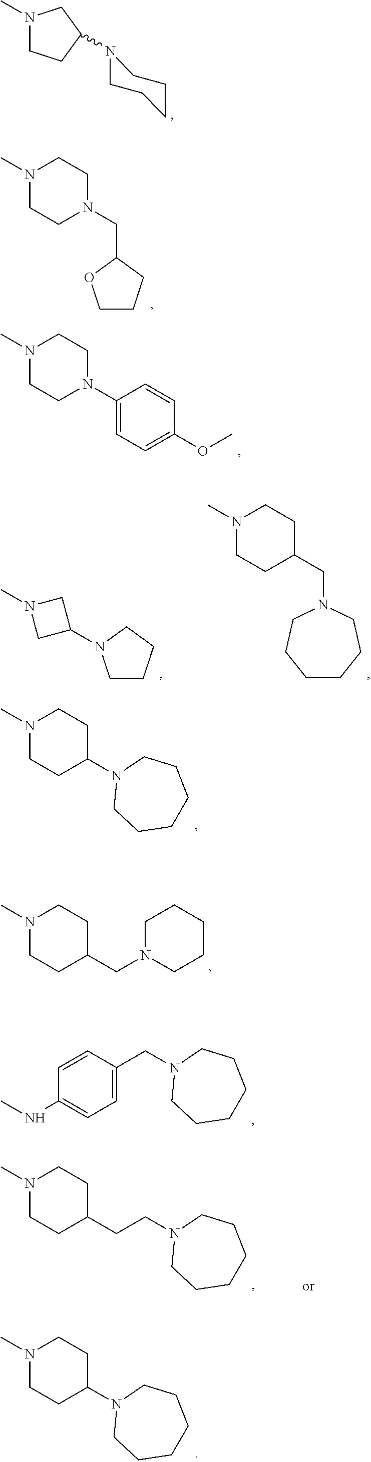

In any of the embodiments described herein, Ring A, Ring B or Ring C may be further substituted in addition to the bonds to the other rings (i.e. Ring A, Ring B or Ring C), to W or to the pyrimidine ring of Formulae I, Ia or Ib.

In still other embodiments, the compound is a compounds of Table 1, or a pharmaceutically acceptable salt, derivative or prodrug thereof:

TABLE 1

| Compound | |
|---|---|
| No. | Structure |
| 1 | 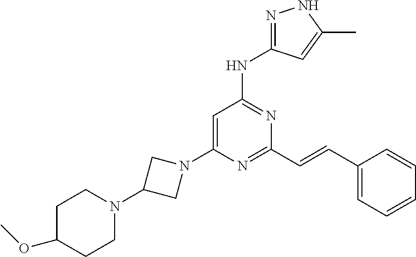 |
| 2 | 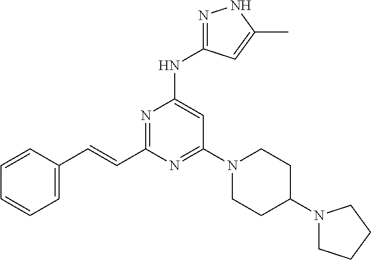 |
| 3 | 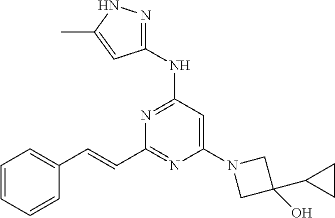 |
| 4 | 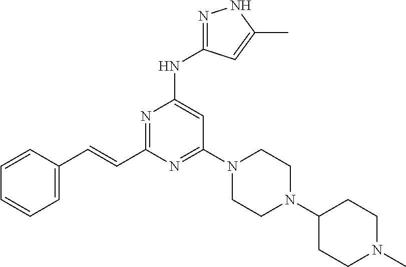 |
| 5 | 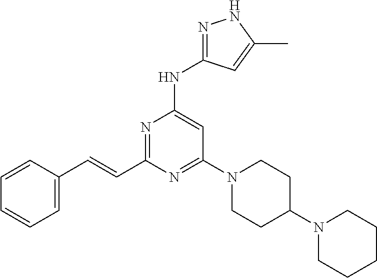 |
| 6 | 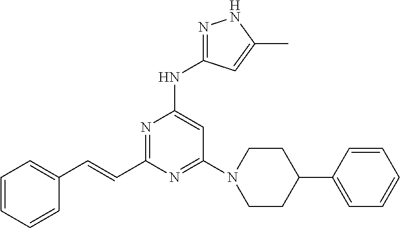 |
| 7 | 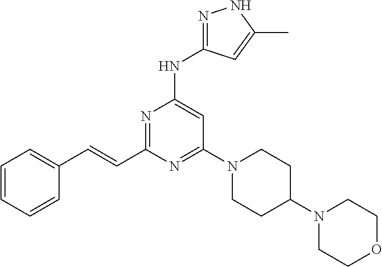 |
| 8 | 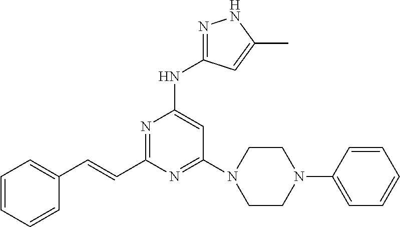 |
| 9 | 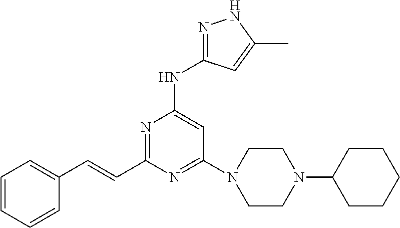 |
| 10 | 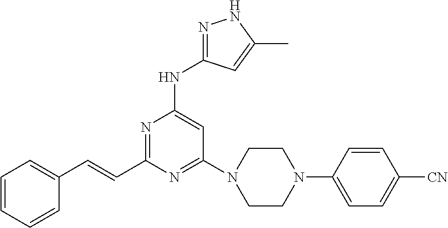 |
| 11 | 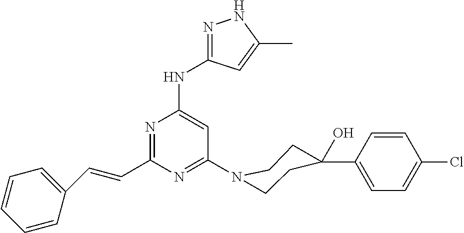 |
| 12 | 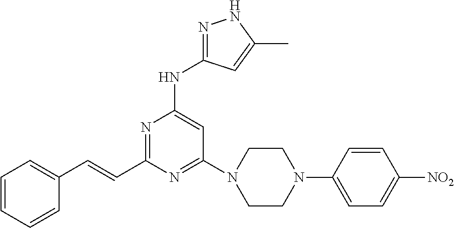 |
| 13 | 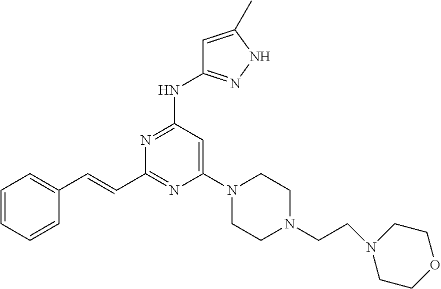 |
| 14 | 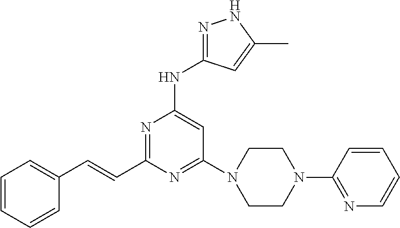 |
| 15 | 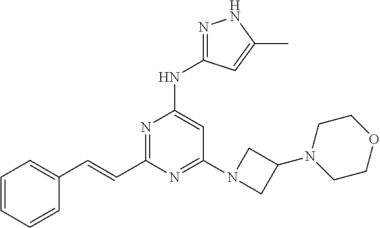 |
| 16 | 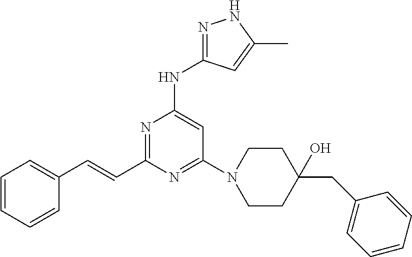 |
| 17 | 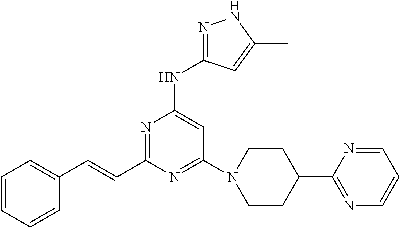 |
| 18 | 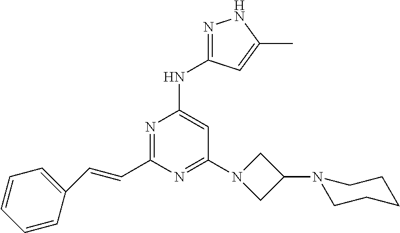 |
| 19 | 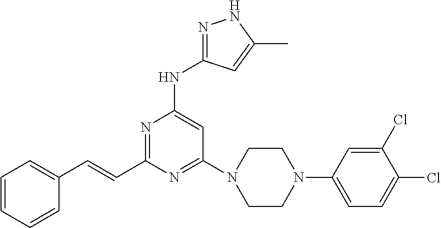 |
| 20 | 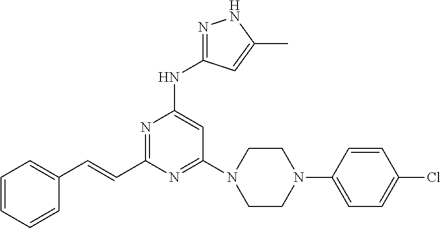 |
| 21 | 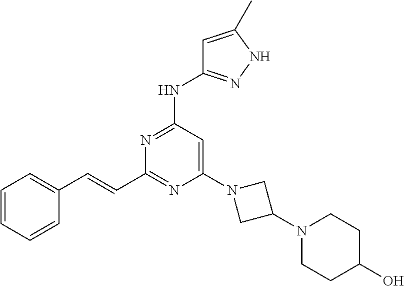 |
| 22 | 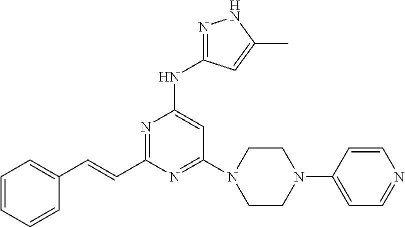 |
| 23 | 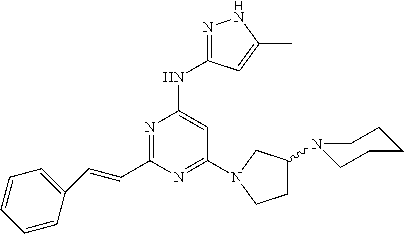 |
| 24 | 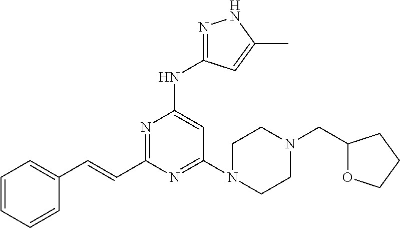 |
| 25 | 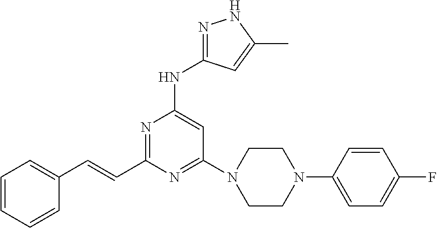 |
| 26 | 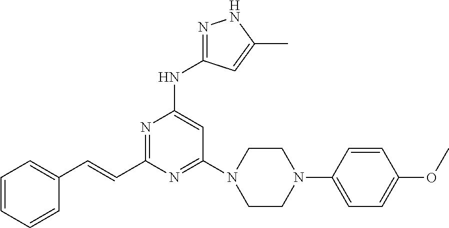 |
| 27 | 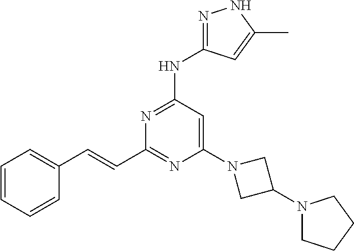 |
| 28 | 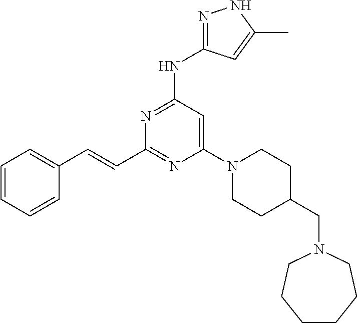 |
| 29 | 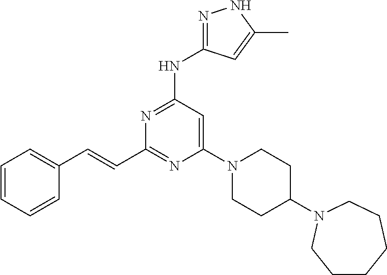 |
| 30 | 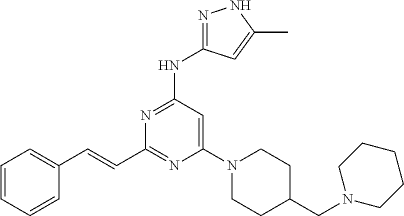 |
| 31 | 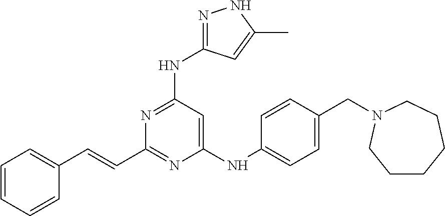 |
| 32 | 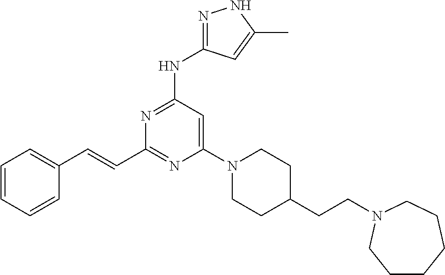 |
| 33 | 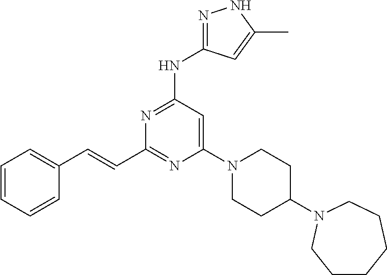 |

In one embodiment, this invention provides a composition comprising a compound of Formulae I, Ia or Ib, and a pharmaceutically acceptable carrier, adjuvant or vehicle. In some such embodiments, the composition is for treating or preventing a kinase mediated disorder.

In one embodiment, the carrier is suitable for oral, parenteral, inhalation, topical, or intradermal administration.

In another embodiment, the composition is incorporated into a biodegradable or non-biodegradable polymer.

In still another embodiment, the composition of comprises a compound of Formulae I, Ia or Ib and an additive. The additive may be selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

In another embodiment, this invention relates to a method of treating or preventing a kinase mediated disease, which method comprises administering to a patient in need of such a treatment a therapeutically effective amount of a compound of Formula I, or a pharmaceutical composition thereof.

In some aspects of the aforementioned methods and compositions, the disorder is mediated by Aurora A, Aurora B, CDK-2, ERK-2, AKT, Src, Lck, Abl, cKit, Flt3, or KDR. In other aspects, the disorder is mediated by Aurora A, Src, Lck, Abl, cKit, Flt3, or KDR.

In one embodiment, a method of treating a patient with a cancer is provided comprising administering to the patient having the cancer an effective cancer-treating amount of a compound of Formulae I, Ia or Ib.

In another embodiment, the a method of treating a patient with a cancer is provided, wherein the cancer is a solid tumor, blood borne tumor, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity, pharynx, lip, tongue, mouth, pharynx, small intestine, colon-rectum, large intestine, rectum, brain and central nervous system, or leukemia.

In still another embodiment, a method of treating a patient with a disease associated with undesirable neovascularization is provided comprising administering to the patient with the undesirable neovascularization an effective amount of a composition comprising a compound of Formulae I, Ia or Ib.

In another embodiment, the disease associated with undesirable neovascularization comprises ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, or corneal graph rejection, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, or post-laser complications.

Another aspect of this invention relates to a method of inhibiting Aurora A activity in a patient, which method comprises administering to the patient a compound of Formulae I, Ia or Ib, or a composition comprising said compound.

Another aspect of this invention relates to a method of treating or preventing a GSK-3-mediated disease with a GSK-3 inhibitor, which method comprises administering to a patient in need of such a treatment a therapeutically effective amount of a compound of Formulae I, Ia or Ib, or a pharmaceutical composition thereof.

Another embodiment comprises a method of treating a patient with an inflammatory disease associated with inflammation comprising administering to the patient with the inflammatory disease an effective amount of a compound of Formulae I, Ia or Ib. The inflammatory disease may be excessive or abnormal stimulation of endothelial cells, atherosclerosis, vascular malfunctions, abnormal wound healing, inflammatory and immune disorders, Bechet's disease, gout or gouty arthritis, abnormal angiogenesis accompanying rheumatoid arthritis, skin diseases, psoriasis, diabetic retinopathy, retinopathy of prematurity, retrolental fibroplasic), macular degeneration, corneal graft rejection, neovascular glaucoma or Osler Weber syndrome.

In still another embodiment, a method of treating patient with a GSK-3 mediated disease is provided comprising administering to the patient with the GSK-3 mediated disease an effective amount of a compound of Formulae I, Ia or Ib. In some embodiments, the GSK-3 mediated disease is diabetes, Alzheimer's disease, Huntington's Disease, Parkinson's Disease, AIDS-associated dementia, amyotrophic lateral sclerosis (AML), multiple sclerosis (MS), schizophrenia, cardiomycete hypertrophy, reperfusion/ischemia, or baldness.

In some embodiments, the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

One aspect of this invention relates to a method of enhancing glycogen synthesis and/or lowering blood levels of glucose in a patient in need thereof, which method comprises administering to the patient a therapeutically effective amount of a compound of Formulae I, Ia or Ib, or a pharmaceutical composition thereof. This method is especially useful for diabetic patients. Another method relates to inhibiting the production of hyperphosphorylated Tau protein, which is useful in halting or slowing the progression of Alzheimer's disease. Another method relates to inhibiting the phosphorylation of beta-catenin, which is useful for treating schizophrenia.

Another aspect of this invention relates to a method of inhibiting GSK-3 activity in a patient, which method comprises administering to the patient a compound of Formulae I, Ia or Ib, or a composition comprising said compound.

Another aspect of this invention relates to a method of treating or preventing a Src-mediated disease with a Src inhibitor, which method comprises administering to a patient in need of such a treatment a therapeutically effective amount of a compound of Formulae I, Ia or Ib, or a pharmaceutical composition thereof.

Another aspect of the invention relates to inhibiting Src activity in a patient, which method comprises administering to the patient a compound of Formulae I, Ia or Ib, or a composition comprising said compound.

Another method relates to inhibiting Aurora A, GSK-3, or Src activity in a biological sample, which method comprises contacting the biological sample with the Aurora A, GSK-3, or Src inhibitor of Formulae I, Ia or Ib, or a pharmaceutical composition thereof, in an amount effective to inhibit Aurora-2, GSK-3, or Src.

Each of the aforementioned methods directed to the inhibition of Aurora A, GSK-3, or Src, or the treatment of a disease alleviated thereby, is carried out with a compound of Formulae I, Ia or Ib, as described above.

The present invention also relates to the processes for preparing the compounds of the invention and to the synthetic intermediates useful in such process, as described below and in the Examples.

### Synthetic Processes

Schemes 1 and 2 below and the experimental description in the examples outline the synthetic methods used to prepare the compounds of the present invention. The variables in Schemes 1 and 2 are as described in the schemes and above for the compound of Formula I. It is understood that the synthetic transformations outlined below can be carried out with a variety of alternate reagents that function to achieve the desired reaction.

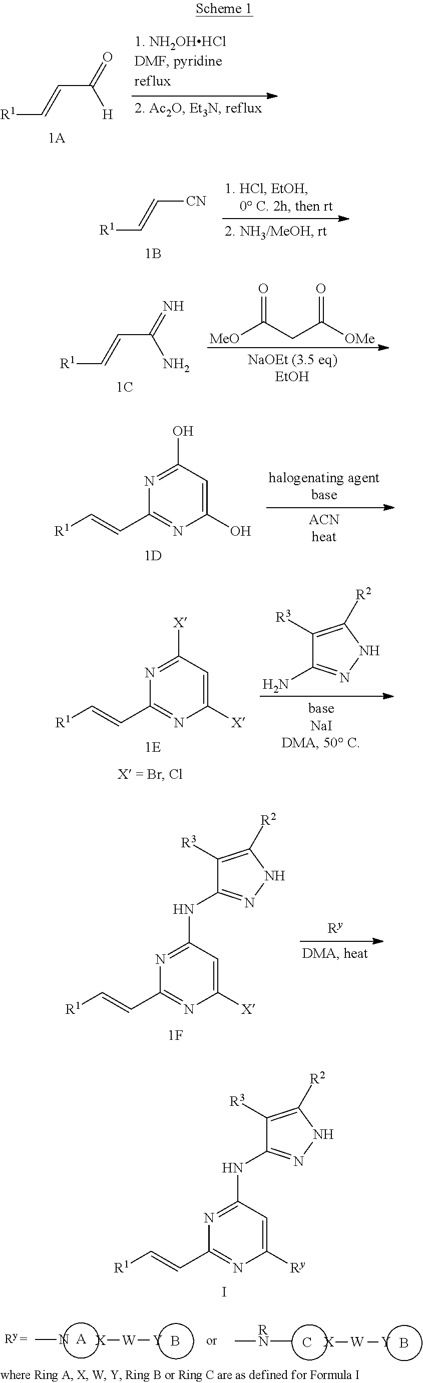

Compounds of Formulae I, Ia or Ib can be synthesized as shown in Scheme 1 and examples of which are indicated by Table 1. When 1B is not commercially available, aldehyde 1A can be converted to nitrile 1B by a two step process (as described in Hilton et al Org. Lett. 2000, 2, 2639). The aldehyde is first converted to the corresponding oxime with hydroxylamine hydrochloride in ethanol. The resulting oxime is converted to the corresponding nitrile via an elimination reaction, using for example, acetic anhydride and triethylamine to give 1B. Nitrile 1B is converted to an amidine via a Pinner reaction using, for example, anhydrous ethanol and dry HCl gas to give the corresponding ethyl amidate as an intermediate, which is then converted to amidine 1C under basic conditions with, for example, methanolic ammonia or ammonium chloride and sodium methoxide. Pyrimidinone 1D is prepared by condensation of 1C, under basic conditions, with a reagent such as dimethylmalonate. Pyrimidinone 1D can be converted to 4,6-dihalogen pyrimidine 1E using a halogenating reagent and a base. In one embodiment, the halogenating reagent is POCl₃ and the base is diisopropyl ethyl amine. The reaction can be carried out with or without the presence of an appropriate solvent, such as acetonitrile. Dihalogenpyrimidine 1E can be substituted with a primary amine, including 3-amino-5-methyl-pyrazole to give pyrimidine 1F. This substitution reaction can be done in a polar aprotic solvent including, for example, dimethylacetamide with a base, including diisopropylethylamine and optionally a catalyst, including NaI. Pyrimidine I is prepared by heating pyrimidine 1F with the desired substituted heterocycle R^{y} including, for example, 4-(1-methylpiperidin-4-yl)piperazine, either neat or in a high boiling aprotic solvent, including dimethyl acetamide. It will be apparent to one of skill in the art that use of a different R^{y} group, such as those including Ring C, will provide the corresponding product.

Scheme 2 can also be used to prepare pyrimidine analogs. In the first step, the most reactive halogen of 2,4,6 trichloropyrimidine (2A) can be replaced by an aminopyrazole to give pyrimidine 2B. The reaction can be done at room temperature in a solvent such as DMA and an added base such as N,N-diisopropyl ethylamine. In the second step, a halogen is replaced with a heterocyclic amine group (R^{y}) to give pyrimidine 2C. Regioisomers of 5C are possible and can be separated by standard purification techniques such as chromatography or crystallization. The last step of Scheme 5 uses Suzuki coupling conditions to couple 2C with the desired vinyl boronic acid or vinyl boronic ester to yield 2D. This reaction typically uses a palladium catalyst, a base and solvent, and can be done at elevated temperatures or in a microwave reactor (for a general reference on the Suzuki Reaction and other named reactions see: Laszlo Kurti, Barbara Czako “Strategic Applications of Named Reactions in Organic Synthesis” Elsevier Academic Press, NY, N.Y. 2005).

Alternative coupling reactions known in the art with an aryl halide and an alkene equivalent may also be used to produce the desired products. For example, rather than a vinyl boronic acid, styrene may be used with a palladium catalyst and a suitable aryl halide to produce the desired product.

These and other standard organic synthesis reactions described herein are described in March's “Advanced Organic Chemistry” 5^{th} Edition, Wiley-Interscience NY, N.Y., 2001, pp. 1552, 1188, 1652-1653 and 1687 respectively.

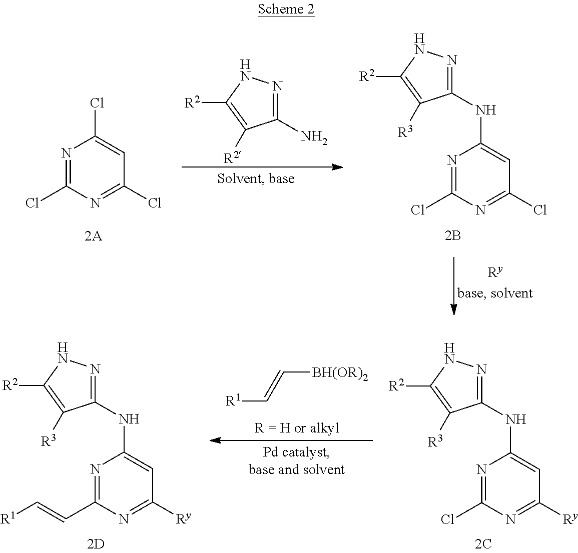

Where necessary in any of the synthetic procedures described herein, appropriate protecting groups may be used. Examples of protection groups can be found in the literature including “Protective Groups in Organic Synthesis—Third Edition” (T. W. Greene, P. G. M. Wuts, Wiley-Interscience, New York, N.Y., 1999). The present invention will be understood more readily by reference to the following examples, which are provided by way of illustration and are not intended to be limiting of the present invention.

### EXAMPLES

The following abbreviations are used in the examples:
ATP: adenosine triphosphate
ACN: Acetonitrile
Brij-35: polyoxyethyleneglycol dodecyl ether
° C.: degrees Celcius
DMEM: Dulbecco's Modified Eagle's Medium
DIPEA: Diisopropylethylamine
DMA: N,N-dimethylacetamide
DMF N,N-dimethylformamide
DMSO: dimethylsulfoxide
DTT: dithiothreitol
EtOAc: ethyl acetate
g: gram
h: hour
H¹ NMR: proton nuclear magnetic resonance
HEPES: 4-(2-hydroxyethyl)piperazine-1-ethanesulfonic acid
Hz; Hertz
hplc: high performance liquid chromatography
IC₅₀ value: concentration of an inhibitor that causes a 50% reduction in a measured activity.
mg: milligram
MHz: megaHertz
mL: milliliter
mmol: millimole
MS: mass spectrum
M/e: mass to charge ratio
Pz: optionally modified or substituted or fused pyrazole ring system
Pet ether: petroleum ether
ppt: precipitation
Rf: ratio to front (ratio of distance traveled by substance/distance traveled by solvent)
SRB: sulphorhodamine-B
TCA: trichloracetic acid
THF: tetrahydrofuran
tlc: thin layer chromatography
br: broad
s: singlet
d: doublet
t: triplet
q: quartet
dd: doublet of doublets
m: multi plot
J: coupling constant
RT: room temperature
δ: part per million
   Additional abbreviations used herein are described in The ACS Style Guide. 3rd Edition Edited by Anne M. Coghill and Lorrin Garson. Oxford University Press, New York. 2006. xiv+430 pp. 18×20.5 cm. ISBN 13: 978-0-8412-3999-9.

### Preparation of Intermediate IF-1

Intermediate 1F-1, (R¹=phenyl, R²=Me, R³═H, X′═Cl), was synthesized following the general narrative described in Scheme 1, except starting with commercially available cinnamonitrile, corresponding to intermediate 1B-1 (R¹=phenyl). Cinnamonitrile (70 g) was dissolved in anhydrous toluene (1.19 L) and absolute ethanol (287 ml, 0.91 mol, 9 eq). The clear solution obtained was cooled to −5° C. and dry HCl gas was gently bubbled for 2 hours after which the reaction was sealed and stirred for 15 hours at 0° C. The reaction was worked up by concentrating the mixture under vacuum at 60-65° C. to dryness, then titurating the solid with ˜1000 mL of hexanes. The white ppt was isolated by vacuum filtration to provide O-ethyl imidate HCl salt (100 gm); ¹H-NMR (200 MHz, DMSO-D₆): 11.63 (2H, bs), 7.98 (1H, d, J=16.2 Hz), 7.73-7.32 (5H, m), 7.01 (1H, d, J=16.2 Hz), 4.46 (2H,q), 1.41 (3H, t, J=5.2 Hz). O-Ethyl imidate HCl salt (100 g) in ethanol (500 ml) was cooled to 0° C. and a methanol solution of dry ammonia (204 ml, 7N, 0.30 mol) was added. The mixture was stirred at room temperature for 12 hours. The reaction mixture was concentrated under reduced pressure and the resultant semisolid was suspended in anhydrous MeOH (˜1000 mL) and acidified with 1.25 N HCl in MeOH. The mixture was concentrated under reduced pressure to provide crude HCl salt. The material was suspended in absolute EtOH (˜1500 mL) and the solid NH₄Cl was filtered. The filtrate was concentrated to provide intermediate 1C-1 (R¹=phenyl, 80 g, 89%); ¹H-NMR (200 MHz, DMSO-D₆): 9.32 (2H, bs), 8.84 (2H, bs), 7.97 (1H, d, J=16.2 Hz), 7.62-7.44 (5H, m), 6.81 (1H, d, J=16.2 Hz); M+147 (100%, m/z).

To intermediate 1C-1 (80 g) in methanol (80 ml) was added dimethyl malonate (80 ml, 1.1 eq) at room temperature. The mixture was cooled to 0° C. and NaOCH₃ (44 g, 4.4 eq) was added slowly over 10 minutes. The light yellow-white solution was heated to 90° C. and refluxed for 4 hours. The reaction was cooled to rt, and solvent was removed under reduced pressure to dryness. The crude product was diluted with water and acidified to pH=2-3 with 1N HCl. The yellow ppt was collected by vacuum filtration, and washed with water ˜1200 mL) then ether (˜1200 mL) and dried under high vacuum for 12 h to provide intermediate 1D-1 (R¹=phenyl, 70 g, 60%); ¹H-NMR (200 MHz, DMSO-D₆): 11.62 (2H, bs), 7.87 (1H, d, J=16.2 Hz), 7.60-7.41 (5H, m), 6.85 (1H, d, J=16.2 Hz), 5.22 (1H, s); M+215 (100%, m/z).

To intermediate 1D-1 (70 g) was slowly added in portions 600 ml of POCl₃. The mixture was stirred for 4-6 hrs at 100° C. The reaction mixture was concentrated under reduced pressure at 60° C. until dryness. The yellow residue was resuspended in ethyl acetate (2000 mL) and cautiously poured into ice water (1000 mL) and the mixture was stirred vigorously for 10 min. The layers were separated, and the organic layer was washed with 1N HCl (3×100 mL), brine (2×1000 mL), dried with sodium sulfate filtered and concentrated under reduced pressure and dried under high vacuum to give 1E-1 (R¹=phenyl, X′═Cl, 70 g, 85%); ¹H-NMR (200 MHz, DMSO-D₆): 7.96 (1H, d, J=16.2 Hz), 7.80 (2H, m), 7.45-7.21 (5H, m); M+250.9, 252.9 (100%, m/z).

To a solution of intermediate 1E-1 (70 g, 0.2278 mole) in anhydrous DMA (400 ml) was added 5-methyl-3aminopyrazole (32.50 g, 0.334 mol), NaI (62.33 g, 0.418 mole) and DIPEA (54 g, 1.48 mol) and the mixture was stirred at 50° C. until reaction was complete by TLC. The reaction mixture was cooled to rt, and diluted with ethylacetate and water. The organic layer was separated and washed with water (2000 mL), NaHCO₃ (2×300 mL, satd), brine (300 mL), dried with sodium sulfate, filtered then concentrated under reduced pressure to yield crude product. The final product was isolated by resuspending the semisolid in DCM:Hexanes (600 mL, 1:1 ratio) with rapid stirring the resulting ppt was isolated wby vacuum filtration and washed with DCM:Hexanes (100 mL, 1:1 ratio) and dried under high vacuum to provide Intermediate 1F-1 (50 g, 57%); ¹H-NMR (200 MHz, DMSO-D₆): 12.12 (1H, s), 10.18 (1H, s), 7.83 (1H, d, J=16.2 Hz), 7.71 (3H, m), 7.45-7.33 (3H, m), 7.12 (1H, d, J=16.2 Hz), 6.22 (1H, bs), 2.21 (3H, s); M+312 (100%, m/z).

### Example 1

### (E)-6-(3-(4-methoxypiperidin-1-yl)azetidin-1-yl)-N-(5-methyl-1H-pyrazol-3-yl)-2-styrylpyrimidin-4-amine

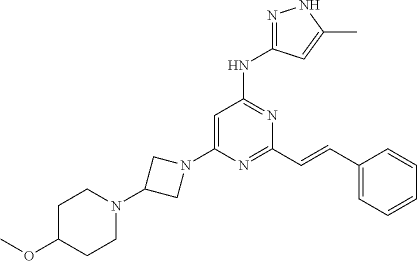

Intermediate 1F-1 (106.5 mg, 0.34 mmol) was dissolved in N,N-dimethylacetamide (5 mL, anhydrous) and 1-(3-Azetidinyl)-4-methoxy piperidine.2HCl (241 mg, 0.99 mmol, 2.5 eq) and di-isopropylethyl amine (333 μL, 1.92 mmol, 5.6 eq) were then added. The mixture was heated to 100° C. under Argon for 18 hrs. The reaction was cooled to RT, and diluted with water (20 mL), and washed with ethyl acetate (3×20 mL). The combined organic layers were washed with NaHCO₃ (satd, 2×30 mL), brine (30 mL) and dried with sodium sulfate. The solution was filtered and the solvent was removed under reduced pressure. The product was purified using an ISCO Combiflash silica column with a CHCl₃:MeOH gradient (0.5% to 10% MeOH) to give (E)-6-(3-(4-methoxypiperidin-1-yl)azetidin-1-yl)-N-(5-methyl-1H-pyrazol-3-yl)-2-styrylpyrimidin-4-amine 54 mg (38% yield). ¹H-NMR (300 MHz, CDCl₃) δ 7.78 (1H, br s), 7.82 (1H, d, J=16 Hz), 7.60-7.57 (2H, m), 7.39-7.24 (3H, m), 6.99 (1H, d, J=16 Hz), 5.92 (2H, s), 4.12-3.99 (2H, m), 3.95-3.83 (2H, m), 3.32 (3H, s), 3.29-3.18 (1H, m), 2.71-2.57 (2H, m), 2.24 (3H, s), 2.15-2.02 (2H, m), 1.96-1.83 (2H, m), 1.67-1.53 (2H, m).

### Example 2

### (E)-N-(5-methyl-1H-pyrazol-3-yl)-6-(4-(pyrrolidin-1-yl)piperidin-1-yl)-2-styrylpyrimidin-4-amine

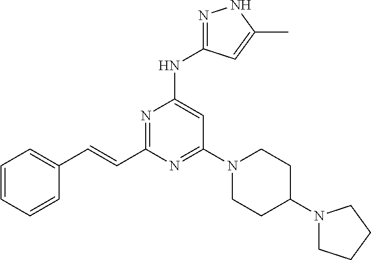

Using the above procedure (no diisopropylethyl amine was added in this case), N-(5-methyl-1H-pyrazol-3-yl)-6-(4-(pyrrolidin-1-yl)piperidin-1-yl)-2-styrylpyrimidin-4-amine was prepared in 37% yield (51 mg) from intermediate 4 (100 mg 0.32 mmol) and 4-(1-pyrrolidinyl)piperidine (148 mg, 0.96 mmol). ¹H-NMR (300 MHz, CDCl3): 7.83 (1H, d, J=15.9 Hz), 7.60 (2H, d, J=7.0 Hz), 7.42-7.29 (3H, m), 6.99 (1H, d, J=15.9 Hz), 6.46 (1H, bs), 5.88 (1H, bs), 4.51 (2H, d, J=13.2 Hz), 2.98-2.82 (6H, m), 2.71 (1H, bs), 2.32 (3H, s), 2.16-2.03 (2H, m), 1.95 (4H, m), 1.81-1.62 (2H, m).

### Example 3

### (E)-3-cyclopropyl-1-(6-(5-methyl-1H-pyrazol-3-ylamino)-2-styrylpyrimidin-4-yl)azetidin-3-ol

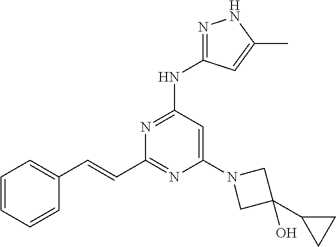

Using the above procedure (iPrOH was reaction solvent), (E)-3-cyclopropyl-1-(6-(5-methyl-1H-pyrazol-3-ylamino)-2-styrylpyrimidin-4-yl)azetidin-3-ol was prepared in 61% yield (113 mg) from intermediate 4 (152 mg 0.48 mmol) and 3-cyclopropylazetidin-3-ol hydrochloride (108 mg, 0.72 mmol). Chromatography was omitted, and final purification was accomplished by precipitation with ethyl acetate and hexanes. ¹H-NMR (300 MHz, CDCl₃) δ 7.79 (1H, d, J=16 Hz), 7.60-7.53 (2H, m), 7.40-7.26 (3H, m), 6.94 (1H, d, J=16 Hz), 6.08-5.87 (2H, m), 3.94-3.76 (4H, m), 2.27(3H, s), 1.31-1.17 (1H, m), 0.58-0.46 (2H, m), 0.46-0.36 (2H, m).

### Example 4

### (E)-N-(5-methyl-1H-pyrazol-3-yl)-6-(4-(1-methylpiperidin-4-yl)piperazin-1-yl)-2-styrylpyrimidin-4-amine

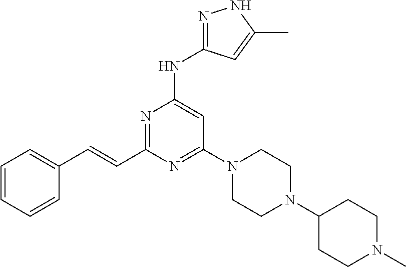

Using the above procedure (no diisopropylethyl amine was added in this case), N-(5-methyl-1H-pyrazol-3-yl)-6-(4-(1-methylpiperidin-4-yl)piperazin-1-yl)-2-styrylpyrimidin-4-amine was prepared in 26% yield (39 mg) from intermediate 4 (100 mg 0.32 mmol) and 1-(1-methyl-4-piperidinyl)piperazine (176 mg, 0.96 mmol). ¹H-NMR (300 MHz, CDCl3): 9.65 (1H, bs), 7.83 (1H, d, J=15.9 Hz), 7.60 (2H, d, J=7.0 Hz), 7.41-7.30 (3H, m), 7.07 (1H, bs), 6.99 (1H, d, J=15.9 Hz), 6.41 (1H, bs), 5.89 (1H, bs), 3.68 (4H, m), 2.95 (2H, d, J=11.6 Hz), 2.64 (4H, t, J=4.8 Hz), 2.32 (3H, s), 2.30 (3H, s), 2.10-1.90 (2H, m), 1.89-1.76 (2H, m), 1.75-1.58 (2H, m).

### Example 5

### (E)-6-(1,4′-bipiperidin-1′-yl)-N-(5-methyl-1H-pyrazol-3-yl)-2-styrylpyrimidin-4-amine

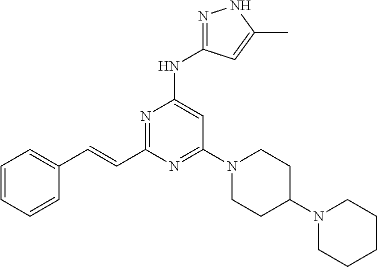

Using the above procedure (no diisopropylethyl amine was added in this case), N-(5-methyl-1H-pyrazol-3-yl)-6-(4-(piperidin-1-yl)piperidin-1-yl)-2-styrylpyrimidin-4-amine was prepared in 53% yield (76 mg) from intermediate 4 (100 mg 0.32 mmol) and 4-piperidinopiperidine (162 mg, 0.96 mmol). ¹H-NMR (300 MHz, CDCl3): 7.84 (1H, d, J=15.9 Hz), 7.63 (2H, d, J=6.8 Hz), 7.41-7.30 (3H, m), 7.00 (1H, d, J=15.9 Hz), 6.78 (1H, bs), 6.40 (1H, bs), 5.88 (1H, bs), 4.55 (2H, d, J=12.9 Hz), 2.87 (2H, t, J=11.5 Hz), 2.55 (5H, m), 2.33 (3H, s), 1.93 (2H, d, J=12.7 Hz), 1.75-1.40 (8H, m).

### Example 6

### (E)-N-(5-methyl-1H-pyrazol-3-yl)-6-(4-phenylpiperidin-1-yl)-2-styrylpyrimidin-4-amine

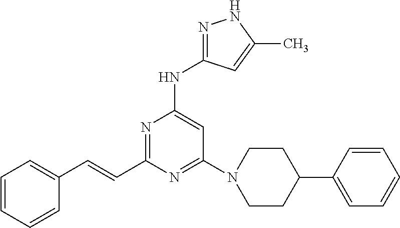

Using the above procedure N-(5-methyl-1H-pyrazol-3-yl)-6-(4-phenylpiperidin-1-yl)-2-styrylpyrimidin-4-amine was obtained (20 mg, 16%) from intermediate 4 and 4-phenyl piperidine (88.6 mg, 0.5 mmol). ¹H-NMR (300 MHz, CDCl₃): 7.89 (1H, d, J=16.2 Hz), 7.63 (2H, d, J=7 Hz), 7.43-7.3 (5H, m), 7.27 (3H, t, J=6 Hz), 7.19 (1H,bs), 7.05 (1H, d, J=16.2 Hz), 6.45 (1H, bs), 5.9 (1H, s), 4.52 (2H, d, J=7 Hz), 3.03 (1H,b), 2.97 (2H,s), 2.80 (1H,b), 2.33 (4H, s) 2.21 (3H, s), 1.99 (2H, b), 1.82 (2H, b).

### Example 7

### (E)-N-(5-methyl-1H-pyrazol-3-yl)-6-(4-morpholinopiperidin-1-yl)-2-styrylpyrimidin-4-amine

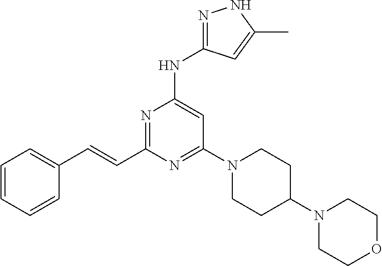

Using the above procedure (no diisopropylethyl amine was added in this case), N-(5-methyl-1H-pyrazol-3-yl)-6-(4-morpholinopiperidin-1-yl)-2-styrylpyrimidin-4-amine was prepared in 62% yield (88 mg) from intermediate 4 (100 mg 0.32 mmol) and 4-morpholinopiperidine (164 mg, 0.96 mmol). ¹H-NMR (300 MHz, CDCl3): 10.10 (1H, bs), 7.84 (1H, d, J=15.9 Hz), 7.60 (2H, d, J=7.3 Hz), 7.78 (1H, bs), 7.43-7.29 (3H, m), 7.00 (1H, d, J=15.9 Hz), 6.41 (1H, bs), 5.95 (1H, bs), 4.47 (2H, d, J=12.9 Hz), 3.71 (4H, m), 2.84 (2H, t, J=11.8 Hz), 2.55 (4H, m), 2.43 (1H, t, J=11.2 Hz), 2.31 (3H, s), 1.91 (2H, d, J=12.1 Hz), 1.58-1.38 (2H, m).

### Example 8

### (E)-N-(5-methyl-1H-pyrazol-3-yl)-6-(4-phenylpiperazin-1-yl)-2-styrylpyrimidin-4-amine

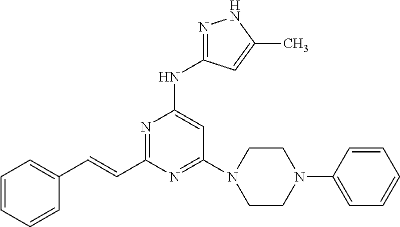

Using the above procedure N-(5-methyl-1H-pyrazol-3-yl)-6-(4-phenylpiperazin-1-yl)-2-styrylpyrimidin-4-amine was obtained (70 mg, 15.9%) from intermediate 4 and phenyl piperazine (188 mg, 1 mmol)—reaction was done on a 1 mmol scale. ¹H-NMR (300 MHz, CDCl₃): 7.91 (1H, d, J=16 Hz), 7.79 (1H, broad), 7.60 (2H, d, J=7 Hz), 7.45-7.33 (3H, m), 7.29-7.23 (3H, m), 7.08 (1H, d, J=16.2 Hz), 6.95 (2H, t, J=7 Hz), 6.89 (1H, d, J=7 Hz), 6.47 (1H, bs), 5.9 (1H, s), 3.80 (4H, t, J=7.0 Hz), 3.25 (4H, t, J=5.0 Hz), 2.35 (3H, s).

### Example 9

### (E)-6-(4-cyclohexylpiperazin-1-yl)-N-(5-methyl-1H-pyrazol-3-yl)-2-styrylpyrimidin-4-amine

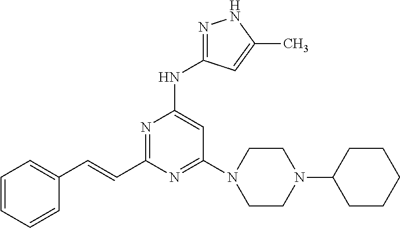

Using the above procedure N-(5-methyl-1H-pyrazol-3-yl)-6-(4-cyclohexylpiperazin-1-yl)-2-styrylpyrimidin-4-amine was obtained (70 mg, 31.6%) from inteiniediate 4 and 1-cyclohexyl piperazine (185.1, 1.1 mmol). ¹H-NMR (300 MHz, DMSO-D₆): 11.95 (1H, s), 9.19 (1H, s), 7.75 (1H, d, J=16 Hz), 7.65 (2H, d, J=7 Hz), 7.45-7.33 (3H, m), 6.95 (1H, d, J=16.0 Hz), 6.65 (1H, bs), 6.00 (1H, s), 3.55 (4H, s), 2.60 (4H, s), 2.21 (3H, s), 1.80 (4H, b), 1.60 (1H, b), 1.35 (4H, b).

### Example 10

### (E)-4-(4-(6-(5-methyl-1H-pyrazol-3-ylamino)-2-styrylpyrimidin-4-yl)piperazin-1-yl)benzonitrile

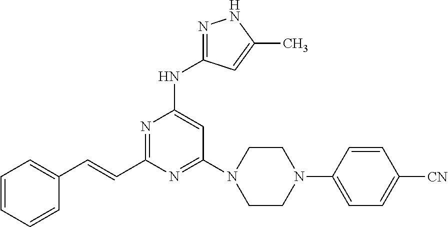

Using the above procedure 4-(4-(6-(5-methyl-1H-pyrazol-3-ylamino)-2-styrylpyrimidin-4-yl)piperazin-1-yl)benzonitrile was obtained (69.9 mg, 30.2%) from intermediate 4 and 4-piperazine benzonitrile (102.8 mg, 0.5 mmol). ¹H-NMR (300 MHz, CDCl₃): 7.89 (1H, d, J=16.2 Hz), 7.63 (2H, d, J=7 Hz), 7.55 (2H, d, J=9 Hz), 7.43-7.33 (3H, m), 7.05 (1H, d, J=16.2 Hz), 6.97 (1H, bs), 6.9 (2H, d, J=9 Hz), 6.47 (1H, bs), 5.9 (1H, s), 3.80-3.70 (4H, m), 3.65-3.45 (4H,m), 2.42 (4H,s), 2.21 (3H, s).

### Example 11

### (E)-4-(4-chlorophenyl)-1-(6-(5-methyl-1H-pyrazol-3-ylamino)-2-styrylpyrimidin-4-yl)piperidin-4-ol

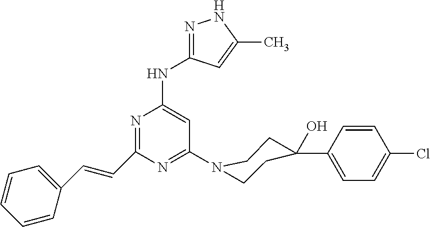

Using the above procedure gave (6-(5-methyl-1H-pyrazol-3-ylamino)-2-styrylpyrimidin-4-yl)-4-(4-chlorophenyl)piperidin-4-ol (120 mg, 60%). was obtained (120 mg, 60%) from intermediate 4 and 4-(4-chlorophenyl)piperidin-4-ol. ¹H-NMR (300 MHz, CDCl₃), 7.85 (1H, d, J=16.1 Hz), 7.60 (2H, d, J=7 Hz), 7.45-7.3 (6H, m), 7.05 (1H, d, J=16.3 Hz), 6.95 (2H, d, J=5.2 Hz), 6.55 (1H, bs), 5.95 (1H, bs), 5.05 (1H, bs), 4.45 (2H, d, J=13.23 Hz), 3.40 (2H, t, J=10.6 Hz), 2.30 (3H, s), 2.08 (3H, m) 1.85 (4H, m).

### Example 12

### (E)-N-(5-methyl-1H-pyrazol-3-yl)-6-(4-(4-nitrophenyl)piperazin-1-yl)-2-styrylpyrimidin-4-amine

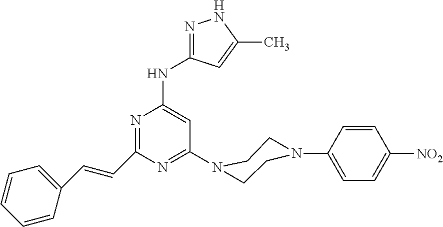

Using the above procedure gave N-(5-methyl-1H-pyrazol-3-yl)-6-(4-(4-nitrophenyl)piperazin-1-yl)-2-styrylpyrimidin-4-amine was obtained (120 mg, 60%) from intermediate 4 and 1-(4-nitrophenyl)piperazine. ¹H-NMR (300 MHz, DMSO-D₆), 11.95 (1H, s), 9.25 (1H, s), 8.10 (2H, d, J=9.4 Hz), 7.78 (1H, d, J=16 Hz), 7.65 (2H, d, J=7.1 Hz), 7.48-7.32 (3H, m), 7.05 (2H, d, J=9.5 Hz), 6.95 (1H, d, J=16.3 Hz), 6.65 (1H, bs), 6.05 (1H, bs), 3.78 (4H, m) 3.68 (4H, m), 2.20 (3H, s).

### Example 13

### (E)-N-(5-methyl-1H-pyrazol-3-yl)-6-(4-(2-morpholinoethyl)piperazin-1-yl)-2-styrylpyrimidin-4-amine

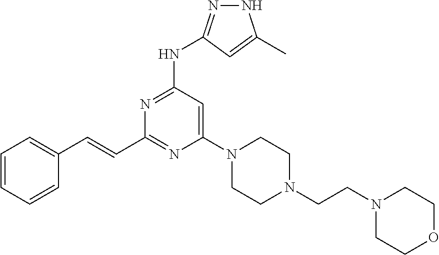

Using the above procedure (no diisopropylethyl amine was added in this case), (E)-N-(5-methyl-1H-pyrazol-3-yl)-6-(4-(2-morpholinoethyl)piperazin-1-yl)-2-styrylpyrimidin-4-amine was prepared in 55% yield (84 mg) from intermediate 4 (99.2 mg 0.32 mmol) and 1-[2-(morpholin-4-yl)ethyl]piperazine (191 mg, 0.96 mmol). ¹H-NMR (300 MHz, CDCl₃) δ 7.91 (1H, br s), 7.83 (1H, d, J=15.5 Hz), 7.61-7.54 (2H, m), 7.42-7.24 (3H, m), 7.0 (1H, d, J=16 Hz), 6.39 (1H, br s), 5.95 (1H, s), 3.73-3.65 (4H, m), 3.65-3.57 (4H, m), 2.58-2.48 (8H, m), 2.48-2.42 (4H, m), 2.27 (3H, s).

### Example 14

### (E)-N-(5-methyl-1H-pyrazol-3-yl)-6-(4-(pyridin-2-yl)piperazin-1-yl)-2-styrylpyrimidin-4-amine

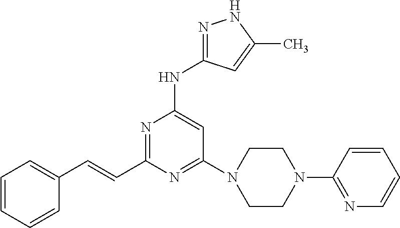

Using the above procedure, N-(5-methyl-1H-pyrazol-3-yl)-6-(4-(pyridin-2-yl)piperazin-1-yl)-2-styrylpyrimidin-4-amine was prepared (35 mg, 16%) from intermediate 4 (156 mg, 0.5 mmol) and 1-(2-pyridyl)piperazine (84 mg, 0.5 mmol). ¹H-NMR (300 MHz, CDCl₃): 8.22 (1H, d, J=3 Hz), 7.95 (1H, broad), 7.85 (1H, d, J=16.2 Hz), 7.60 (2H, d, J=7 Hz), 7.43 (1H, t, J=7 Hz), 7.45-7.33 (3H, m), 7.12 (1H, d, J=16.2 Hz), 6.65 (2H, t, J=7 Hz), 6.4 (1H, bs), 5.9 (1H, s), 3.76 (4H, dd, J=6.0, 3.0 Hz), 3.65 (4H, dd, J=3.0, 4.0 Hz), 2.21 (3H, s).

### Example 15

### (E)-N-(5-methyl-1H-pyrazol-3-yl)-6-(3-morpholinoazetidin-1-yl)-2-styrylpyrimidin-4-amine

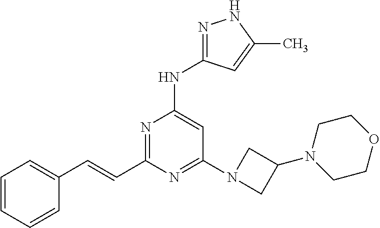

Using the above procedure, N-(5-methyl-1H-pyrazol-3-yl)-6-(3-morpholinoazetidin-1-yl)-2-styrylpyrimidin-4-amine was prepared (19.2 mg, 9.2%) from intermediate 4 and 4-(azetidin-3-yl)morpholine 2HCl (107.5 mg, 0.5 mmol). ¹H-NMR (300 MHz, CDCl₃): 7.85 (1H, d, J=16.2 Hz), 7.65 (1H, bs), 7.60 (2H, d, J=7 Hz), 7.4-7.33 (3H, m), 7.12 (1H, d, J=16.2 Hz), 5.9 (2H, d, J=9 Hz), 4.10 (2H, t, J=7 Hz), 3.95 (2H, t, J=7 Hz), 3.80-3.70 (4H, m), 3.35-3.25 (1H,m), 2.42 (4H,s), 2.21 (3H, s).

### Example 18

### (E)-N-(5-methyl-1H-pyrazol-3-yl)-6-(3-(piperidin-1-yl)azetidin-1-yl)-2-styrylpyrimidin-4-amine

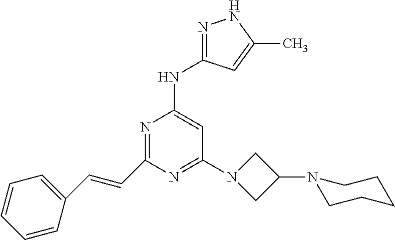

Using the above procedure, N-(5-methyl-1H-pyrazol-3-yl)-6-(3-(piperidin-1-yl)azetidin-1-yl)-2-styrylpyrimidin-4-amine was prepared (120 mg, 60%) from intermediate 4 and 1-(azetidin-3-yl)piperidine. ¹H-NMR (300 MHz, CDCl₃): 7.86 (1H, d, J=16.04 Hz), 7.60 (2H, d, J=7.1 Hz), 7.41-7.32 (3H, m), 7.02 (1H, d, J=16.1 Hz), 6.95 (1H, bs), 5.95 (1H, bs), 5.89 (1H, s) 4.15 (2H, t, 8 Hz), 3.95 (1H, t, J=8 Hz), 3.30 (1H, dd, J=5.3 Hz), 2.42 (4H, m), 2.30 (3H, s), 1.65 (4H, m), 1.48 (2H, m).

### Example 21

### (E)-1-(1-(6-(5-methyl-1H-pyrazol-3-ylamino)-2-styrylpyrimidin-4-yl)azetidin-3-yl)piperidin-4-ol

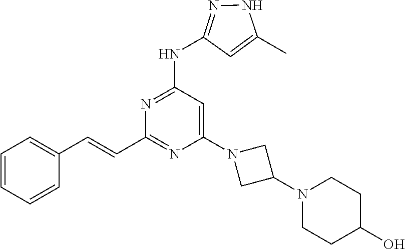

Using the above procedure, (E)-1-(1-(6-(5-methyl-1H-pyrazol-3-ylamino)-2-styrylpyrimidin-4-yl)azetidin-3-yl)piperidin-4-ol was prepared in 39% yield (54 mg) from intermediate 4 (99.2 mg 0.32 mmol) and 1-(3-azetidinyl)-4-piperidinol.2HCl (220 mg, 0.96 mmol). ¹H-NMR (300 MHz, CDCl₃) δ 7.81 (1H, d, J=16 Hz), 7.75 (1H, br s), 7.60-7.52 (2H, m), 7.39-7.25 (3H, m), 6.98 (1H, d, J=16 Hz), 5.90 (1H, br s), 4.10-3.99 (2H, m), 3.94-3.83 (2H, m), 3.77-3.66 (1H, m), 3.29-3.27 (1H, m), 2.73-2.60 (3H, m), 2.24(3H, s), 2.13-1.98 (2H, m), 1.95-1.81 (2H, m), 1.66-1.50 (2H, m).

### Example 22

### (E)-N-(5-methyl-1H-pyrazol-3-yl)-6-(4-(pyridin-4-yl)piperazin-1-yl)-2-styrylpyrimidin-4-amine

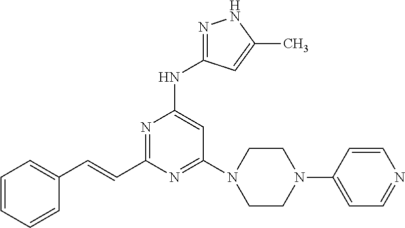

Using the above procedure, N-(5-methyl-1H-pyrazol-3-yl)-6-(4-(pyridin-4-yl)piperazin-1-yl)-2-styrylpyrimidin-4-amine was prepared (20 mg, 9.1%) from intermediate 4 and 1-(pyridin-4-yl)piperazine. ¹H-NMR (300 MHz, DMSO-D₆): 11.95 (1H, s), 9.19 (1H, s), 8.20 (2H, d, J=7 Hz), 7.75 (1H, d, J=16 Hz), 7.66 (2H, d, J=7 Hz), 7.45-7.33 (3H, m), 6.95 (1H, d, J=16.0 Hz), 6.86 (2H, d, J=7 Hz), 6.70 (1H, b), 3.73 (4H, bs), 3.50 (4H, s), 2.21 (3H, s).

### Example 23

### (E)-N-(5-methyl-1H-pyrazol-3-yl)-6-(3-(piperidin-1-yl)pyrrolidin-1-yl)-2-styrylpyrimidin-4-amine

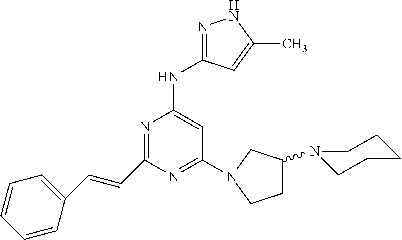

Using the above procedure, (E)-N-(5-methyl-1H-pyrazol-3-yl)-6-(3-(piperidin-1-yl)pyrrolidin-1-yl)-2-styrylpyrimidin-4-amine was prepared (107 mg, 57%) from intermediate 4 and 1-(pyrrolidin-3-yl)piperidine. ¹H-NMR (300 MHz, CDCl₃): 7.86 (1H, d, J=16.1 Hz), 7.62 (2H, d, J=9.2 Hz), 7.41-7.35 (3H, m), 7.05 (1H, d, J=16.2 Hz), 6.91 (1H, bs), 6.01 (1H, bs), 5.95 (1H, s) 3.45 (1H, m), 3.35 (1H, t, J=6.2 Hz), 2.45 (1H, m), 2.52 (4H, m), 2.32 (3H, s), 2.29 (1H, m), 2.0-1.85 (2H, m), 1.65 (4H, m), 1.45 (2H, m), 1.28 (1H, d, J=2.2 Hz), 0.85 (1H, dd, J=6 Hz).

### Example 24

### (E)-N-(5-methyl-1H-pyrazol-3-yl)-2-styryl-6-(4-((tetrahydrofuran-2-yl)methyl)piperazin-1-yl)pyrimidin-4-amine

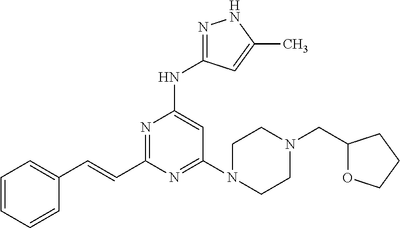

Used same general procedure as Example 7. Product was obtained from reaction between intermediate 4 and 1-tetrahydro-2-fury)methyl piperazine (0.86 ml, 0.5 mmol). Workup and chromatographic purification gave 6-(4-((tetrahydrofuran-2-yl)methyl)piperazin-1-yl)-N-(5-methyl-1H-pyrazol-3-yl)-2-styrylpyrimidin-4-amine (14 mg, 6.5%). ¹H-NMR (300 MHz, CDCl₃): 7.89 (1H, d, J=16.2 Hz), 7.63 (2H, d, J=7 Hz), 7.43-7.3 (3H, m), 7.05 (1H, d, J=16.2 Hz), 6.39 (1H, bs), 5.90 (1H, s), 4.10 (1H, m), 3.90 (1H, m), 3.70 (4H, s), 2.60 (4H, s), 2.51 (1H, d, J=7 Hz), 2.30 (3H, s), 2.00 (1H, m), 1.91 (2H, m), 1.50 (1H, m).

### Biological Testing

### Biological Testing Example 1

### Aurora A (Aurora 2) Inhibition Assay

Compounds were tested for their potency against recombinant Aurora A (Upstate, Lake Placid, N.Y.) using the PanVera Z′-Lyte kinase assay kit-Ser/Thr 1 peptide (Invitrogen, Carlsbad, Calif.). Assays were carried out in kinase assay buffer (50 mM HEPES, pH 7.5, 10 mM MgCl₂, 5 mM EGTA, 0.05% Brij-35, 2 mM DTT). Test compounds were initially dissolved in DMSO at 100× the highest tested concentration, then serially diluted to 4× test concentrations in kinase assay buffer. Next, Aurora A (final concentration 200-500 ng/mL), Z′-Lyte Ser/Thr 1 peptide (final concentration 2 μM) and ATP (final concentration 10 μM) were added according to the manufacturer's instructions. Assays were carried out in half-area 96-well white polystyrene assay plates (Corning, Corning, N.Y.) in a final volume of 20 μl. The reaction was allowed to proceed for 1 h at room temperature in the dark, at which point the development reagent and stop reagent were added according to the manufacturer's instructions. Coumarin (Ex. 400 nm, Em. 465 nm) and fluorescein (Ex. 400 nm, Em. 565 nm) fluorescence values were measured on a SpectraFluor Plus plate reader (Tecan, Durham, N.C.). The emission ratio (coumarin/fluorescein) was determined and used to calculate the percent phosphorylation for each well. Wells containing substrate but no kinase and wells containing a phosphopeptide control were used to set 0% and 100% phosphorylation values, respectively. Typically 20-40% of the substrate was phosphorylated in wells without inhibitor. Dose-response curves of relative Aurora A activity vs. inhibitor concentration were plotted with Grafit (Erithacus Software, Horley, Surrey, UK).

The compounds of the invention were shown to inhibit Aurora A using the method described above. For example, compounds 1, 2, 3, 4, 5, 7, 9, 13 14, 15, 16, 18, 21, 22, 23, 24 and 30, were shown to have IC₅₀ values in this assay of less than or equal to 100 nM, and the compounds 6, 8, 10, 11, and 12 were shown to have IC₅₀ values in this assay of greater than 100 nM and less than or equal to 1 μM.

### Biological Testing Example 2

### Aurora B (Aurora 1) Inhibition Assay

Assays for Aurora B kinase inhibition were carried out similarly to those for Aurora A kinase (see above) with the following modifications. Aurora B kinase (BPS Biosciences, San Diego, Calif.) was used as the enzyme, at a concentration was 2.5 μg/mL. The ATP concentration was 50 μM, and the kinase reaction was allowed to proceed for 16 h. Sodium orthovanadate (20 μM) was added to the buffer to inhibit contaminating phosphatases. The compounds of the invention were shown to inhibit Aurora B using the method described above. For example, compounds 3 and 7, were shown to have IC₅₀ values in this assay of less than or equal to 100 nM, and the compounds 1, 2, 5, 9, 13, 15, 18, 21, 22, 23 and 24 were shown to have IC₅₀ values in this assay of greater than 100 nM and less than or equal to 1 μM.

### Biological Testing Example 3

### Src Kinase Inhibition Assay

Compounds were assayed for Src kinase inhibitory activity using N-terminal His-tagged human Src (Upstate USA Inc, 706 Forest Street, Charlottesville, Va.). Serial dilutions of compound were assayed in a final reaction volume of 25 μL by incubating a solution of the above Src kinase (5-10 mU), 8 mM MOPS (3-(N-morpholino)propanesulfonic acid) pH 7.0, 0.2 mM EDTA (ethylenediamine tetracetic acid), 250 μM amino acid sequence KVEKIGEGTYGVVYK (Upstate USA Inc, 706 Forest Street, Charlottesville, Va.), and 10 mM magnesium acetate and [γ-³³P-ATP] (specific activity of about 500 cpm/pmol, concentration as required). The reaction was initiated by the addition of the magnesium acetate and [γ-³³P-ATP] mixture. After incubation for 40 minutes at room temperature, the reaction was stopped by the addition of 5 μL of a 3% phosphoric acid solution. A 10 μL aliquote of the reaction was then spotted onto a P30 filtermat (PerkinElmer, Wellesley, Mass.) and washed three times for five minutes in 75 mM phosphoric acid and once in methanol prior to drying and scintillation counting. Inhibition of Src activity was determined by comparison to assays that contained no inhibitor. For example, compounds 1, 2, 3, 4, 5, 7, 9, 13, 15, 18, 21, 22, 23, 24 and 30 were shown to have IC₅₀ values in this assay of less than or equal to 100 nM, and the compounds 8, 10, 11, 12, 14 and 16 was shown to have IC₅₀ values in this assay of greater than 100 nM and less than or equal to 1 μM.

### Biological Testing Example 4

### Flt3 Kinase Inhibition Assay

Compounds were assayed for Flt3 kinase inhibitory activity using N-terminal GST-tagged recombinant human Flt3, residues 564-end (Upstate USA Inc, 706 Forest Street, Charlottesville, Va.). Serial dilutions of compound were assayed in a final reaction volume of 25 μL by incubating a solution of the above Flt3 kinase (5-10 mU), 8 mM MOPS (3-(N-morpholino)propanesulfonic acid) pH 7.0, 0.2 mM EDTA (ethylenediamine tetracetic acid), 50 μM amino acid sequence EAIYAAPFAKKK (Upstate USA Inc, 706 Forest Street, Charlottesville, Va.), and 10 mM magnesium acetate and [γ-³³P-ATP] (specific activity of about 500 cpm/pmol, concentration as required). The reaction was initiated by the addition of the magnesium acetate and [γ-³³P-ATP] mixture. After incubation for 40 minutes at room temperature, the reaction was stopped by the addition of 5 μL of a 3% phosphoric acid solution. A 10 μL aliquote of the reaction was then spotted onto a P30 filtermat (PerkinElmer, Wellesley, Mass.) and washed three times for five minutes in 75 mM phosphoric acid and once in methanol prior to drying and scintillation counting. Inhibition of Flt3 activity was determined by comparison to assays that contained no inhibitor. The compounds of the invention were shown to inhibit Flt3 kinase using the method described above. For example, compounds 1, 2, 3, 4, 5, 7, 8, 9, 10, 11, 12, 13, 14, 15, 16, 18, 21, 22, 23, 24 and 30 were shown to have IC₅₀ values in this assay of less than or equal to 100 nM, and the compound 6 was shown to have IC₅₀ values in this assay of greater than 100 nM and less than or equal to 1 μM.

### Biological Testing Example 5

### KDR Kinase Inhibition Assay

Compounds were assayed for KDR kinase inhibitory activity using N-terminal His₆-tagged recombinant human KDR, residues 790-end (Upstate USA Inc, 706 Forest Street, Charlottesville, Va.). Serial dilutions of compound were assayed in a final reaction volume of 25 μL by incubating a solution of the above KDR kinase (5-10 mU), 8 mM MOPS (3-(N-morpholino)propanesulfonic acid) pH 7.0, 0.2 mM EDTA (ethylenediamine tetracetic acid), 0.33 mg/mL myelin basic protein (Upstate USA Inc, 706 Forest Street, Charlottesville, Va.), and 10 mM magnesium acetate and [γ-³³P-ATP] (specific activity of about 500 cpm/pmol, concentration as required). The reaction was initiated by the addition of the magnesium acetate and [γ-³³P-ATP] mixture. After incubation for 40 minutes at room temperature, the reaction was stopped by the addition of 5 μL of a 3% phosphoric acid solution. A 10 μL aliquote of the reaction was then spotted onto a P30 filtermat (PerkinElmer, Wellesley, Mass.) and washed three times for five minutes in 75 mM phosphoric acid and once in methanol prior to drying and scintillation counting. Inhibition of KDR activity was determined by comparison to assays that contained no inhibitor. The compounds of the invention were shown to inhibit KDR kinase using the method described above. For example, compounds 1, 2, 3, 4, 5, 7, 9, 12, 13, 15, 18, 21, 22, 23, 24 and 30 were shown to have IC₅₀ values in this assay of less than or equal to 100 nM, and the compounds 10, 14 and 16 were shown to have IC₅₀ values in this assay of greater than 100 nM and less than or equal to 1 μM.

### Biological Testing Example 6

### Whole Cell Cytotoxicity Assay: Sulforhodamine B

(Reference: Developmental Therapeutics Program NCUNIH; http://dtp.nci.nih.gov/branches/btb/ivclsp.html)

Human tumor-derived cell lines, HCT116 or MCF7 (ATCC) were plated in a 96 well plate in DMEM containing 10% fetal bovine serum and 2 mM L-glutamine at a density of 500 HCT116 cells or 1,000 MCF7 cells per well and incubated at 37° C., 5% CO₂, for 24 hours prior to the addition of experimental compounds. Compounds were added using the dilution series indicated to duplicate plates and the cells were incubated in media plus compound for 96 hours. An additional plate was fixed in 10% TCA at the time of the addition of compound to provide a measurement of the cell population at time zero, the time of drug addition. Following the 96 hour incubation, cells were fixed in situ by gently aspirating off the culture media and then adding 50 ul of ice cold 10% TCA per well and incubation at 4° C. for 60 minutes. The plates were washed with tap water five times and allowed to air dry for 5 minute. 50 ul of a 0.4% (w/v) Sulforhodamine B solution in 1% (v/v) acetic acid was added per well and the cells were incubated for 30 minutes at room temperature. Following staining, plates were washed four times with 1% acetic acid to remove any unbound dye and then allowed to air dry for 5 minutes. The stain was solubilized with 100 ul of 10 mM Tris pH 10.5 per well and placed on an orbital rotator for 5 minutes. The absorbance was read at 570 nm. Percentage growth was calculated using the absorbance readings from the time zero plate (Tz) and the dilution series plate (C) which included a column of cells grown in media without compound as a control (C) using the formulas:

[(Ti−Tz)/(C−Tz)]×100 for concentrations for which Ti>/=Tz

[(Ti−Tz)/Tz]×100 for concentrations for which Ti<Tz.

Three dose response parameters were calculated for each experimental agent. Growth inhibition of 50% (GI50) was calculated from [(Ti−Tz)/(C−Tz)]×100=50, which was the drug concentration resulting in a 50% reduction in the net protein increase (as measured by SRB staining) in control cells during the drug incubation. The drug concentration resulting in total growth inhibition (TGI) was calculated from Ti=Tz. The LC50 (concentration of drug resulting in a 50% reduction in the measured protein at the end of the drug treatment as compared to that at the beginning) indicating a net loss of cells following treatment ws calculated from [(Ti−Tz)/Tz]×100=−50. Values are calculated for each of these three parameters if the level of activity was reached; however, if the effect was not reached or was exceeded, the value for that parameter is expressed as greater or less than the maximum or minimum concentration tested.

The compounds of the invention were shown to inhibit HCT-116 cell growth using the method described above. For example, compounds 5 and 22 were shown to have IC₅₀ values in this assay of less than or equal to 100 nM, and the compound 1, 2, 3, 4, 7, 9, 11, 12 13, 15, 16, 18, 21, 23, 24, and 30 was shown to have IC₅₀ values in this assay of greater than 100 nM and less than or equal to 1 μM.

1. A compound of the Formula I: 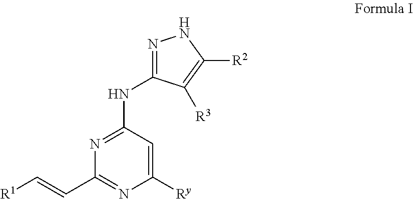
or a pharmaceutically acceptable derivative or prodrug thereof, wherein:
R^{y} is 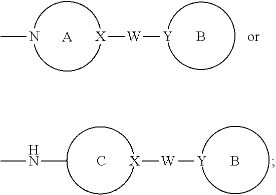
X is N, C or CR;
Y is N, C, or CR;
Ring A is an optionally substituted 4, 5 or 6 membered monocyclic heterocyclic ring;
Ring B is an optionally substituted 3-7 membered monocyclic or 8-10 membered bicyclic ring selected from carbocyclyl, aryl, heterocyclyl, or heteroaryl;
Ring C is an optionally substituted 3, 4, 5 or 6 membered heterocyclic, carbocyclic, aryl or heteroaryl ring;
W is (CR₂)ₙ, where n is 0, 1, 2, 3, 4 or 5;
R¹ is an optionally substituted monocyclic or bicyclic aryl ring;
R² and R³ are independently hydrogen, optionally substituted aliphatic, optionally substituted carbocyclyl, optionally substituted aryl, optionally substituted heteroaryl, optionally substituted heterocyclyl, or R² and R³ taken together with their intervening atoms form a fused, optionally substituted unsaturated or partially unsaturated ring having 0-3 ring heteroatoms; and
R is hydrogen, aliphatic, aryl, aralkyl, alkylaryl, heterocyclyl, heteroaryl, halo, hydroxyl, alkoxy, amino, alkylamino, or dialkylamino.

2. The compound of claim 1, wherein R² is aliphatic.

3. The compound of claim 1, wherein R¹ is phenyl.

4. The compound of claim 1, wherein:
R¹ is phenyl,
R² is aliphatic,
R³ is H, and
R^{y} is 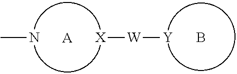
Ring B is an optionally substituted 3-7 membered monocyclic ring selected from carbocyclyl, aryl, heterocyclyl, or heteroaryl.

5. The compound of claim 1, wherein:
R¹ is phenyl,
R² is aliphatic,
R³ is H, and
R^{y} is 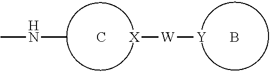
Ring B is an optionally substituted 3-7 membered monocyclic ring selected from carbocyclyl, aryl, heterocyclyl, or heteroaryl.

6. The compound of claim 4, wherein Ring B is optionally substituted carbocyclyl or optionally substituted aryl.

7. The compound of claim 4, wherein Ring B is optionally substituted heterocyclyl or optionally substituted heteroaryl.

8. The compound of claim 6, wherein Ring B is optionally substituted phenyl, optionally substituted cyclopropyl, or optionally substituted cyclohexyl.

9. The compound of claim 7, wherein Ring B is optionally substituted piperidinyl, optionally substituted pyrrolidinyl, optionally substituted pyridinyl, optionally substituted morpholinyl, optionally substituted diazinyl, optionally substituted tetrahydrofuranyl, or optionally substituted azepanyl.

10. The compound of claim 5, wherein Ring C is optionally substituted heterocyclyl or optionally substituted aryl.

11. The compound of claim 8, wherein Ring B is optionally substituted phenyl.

12. The compound of claim 4, wherein Ring A is optionally substituted piperidinyl, optionally substituted pyrrolidinyl, optionally substituted piperazinyl, or optionally substituted azetidinyl.

13. The compound of claim 1, wherein:
X is CR,
W is (CH₂)ₙ;
n is 0, 1, or 2, and
R is hydrogen or hydroxyl.

14. The compound of claim 1, wherein:
X is N;
W is (CH₂)ₙ; and
n is 0, 1, or 2.

15. The compound of claim 1, wherein:
X is C,
W is (CH₂)ₙ; and
n is 0, 1, or 2.

16. The compound of claim 1, wherein the compound has the structure of Formula Ib:

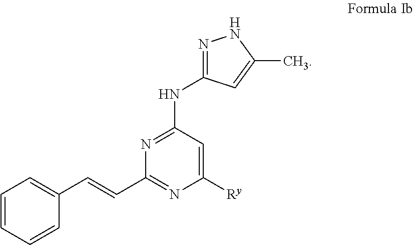

17. The compound of claim 14, wherein Ring B is phenyl, piperidinyl, morpholinyl, pyridinyl, pyrimidinyl, or homopiperidinyl.

18. The compound of claim 5, wherein:
Ring C is phenyl;
W is (CH₂)ₙ, and n is 0, 1, or 2, and
Ring B is optionally substituted heterocyclyl or optionally substituted heteroaryl.

19. The compound of claim 1, wherein the compound is selected from the group consisting of: 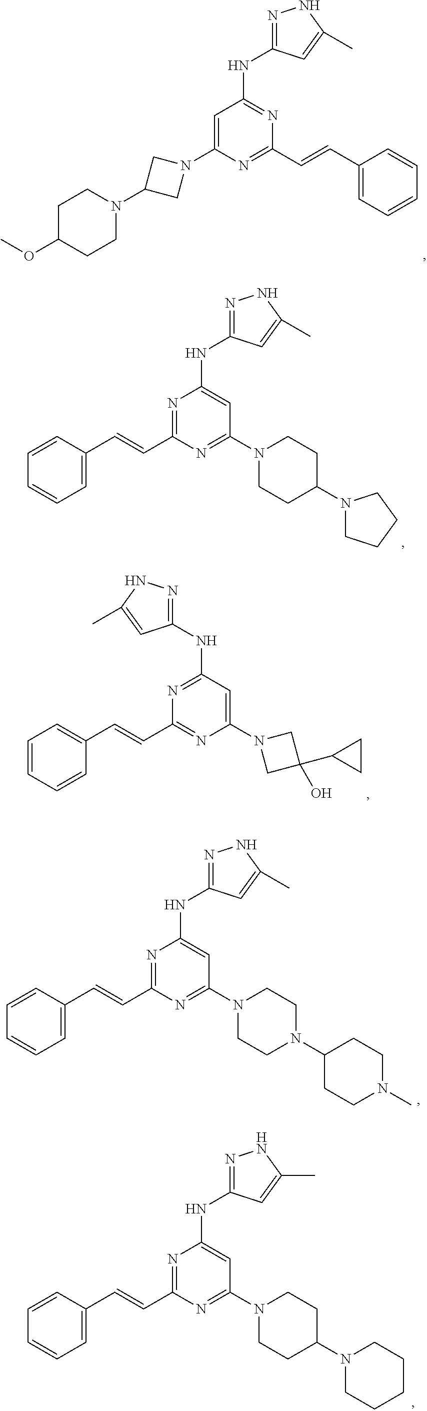 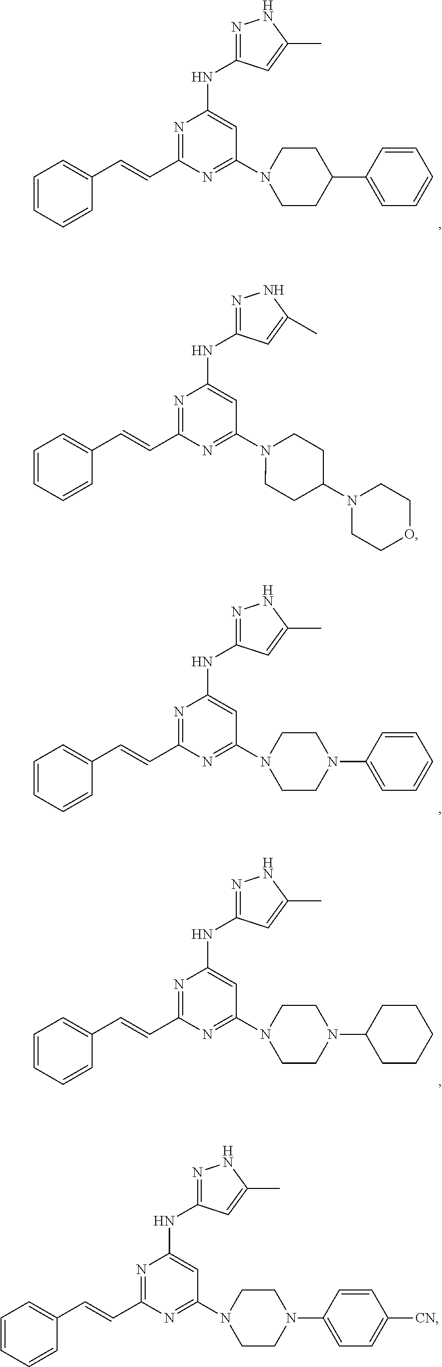 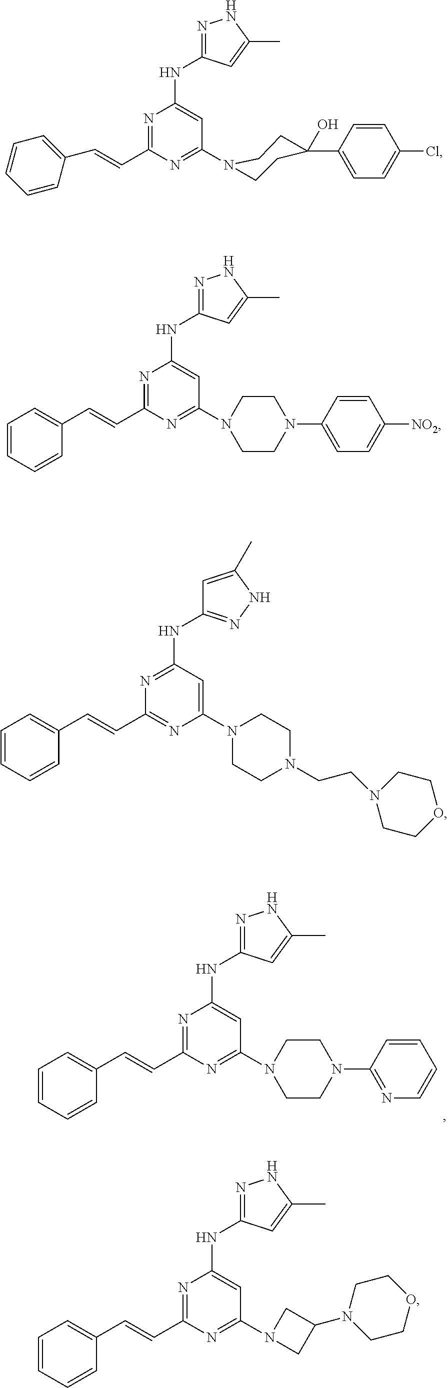 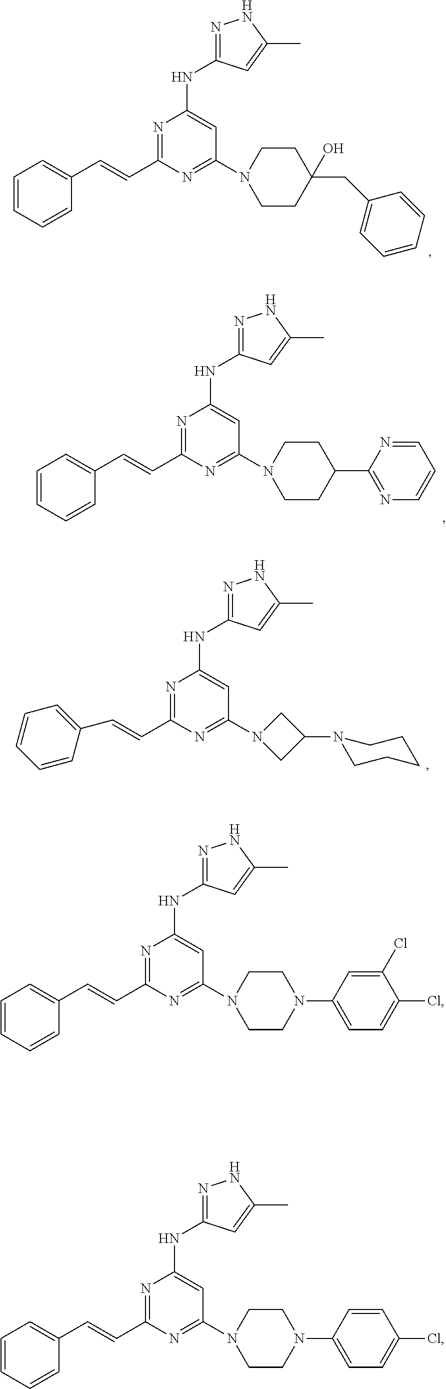 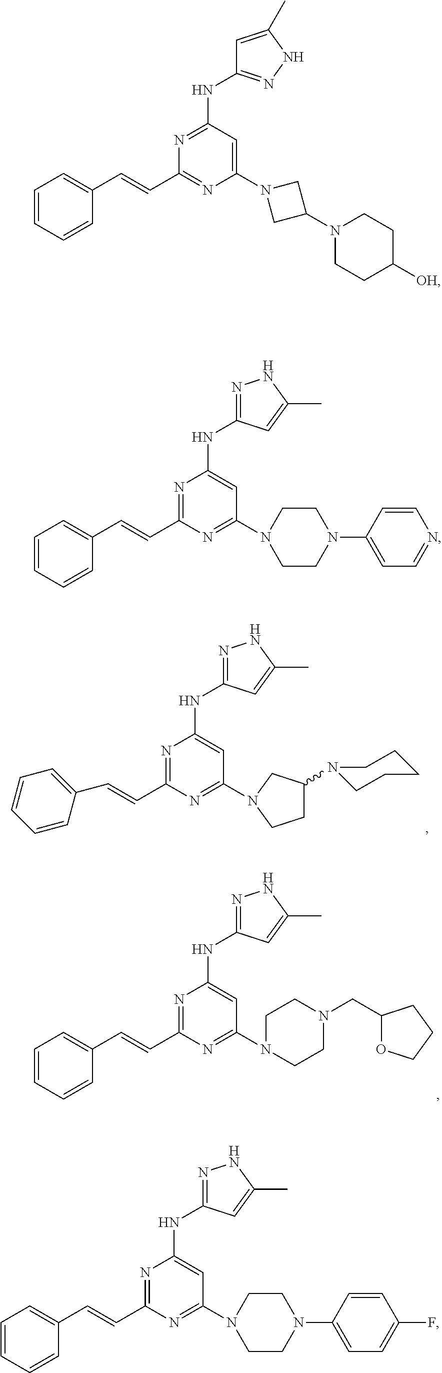 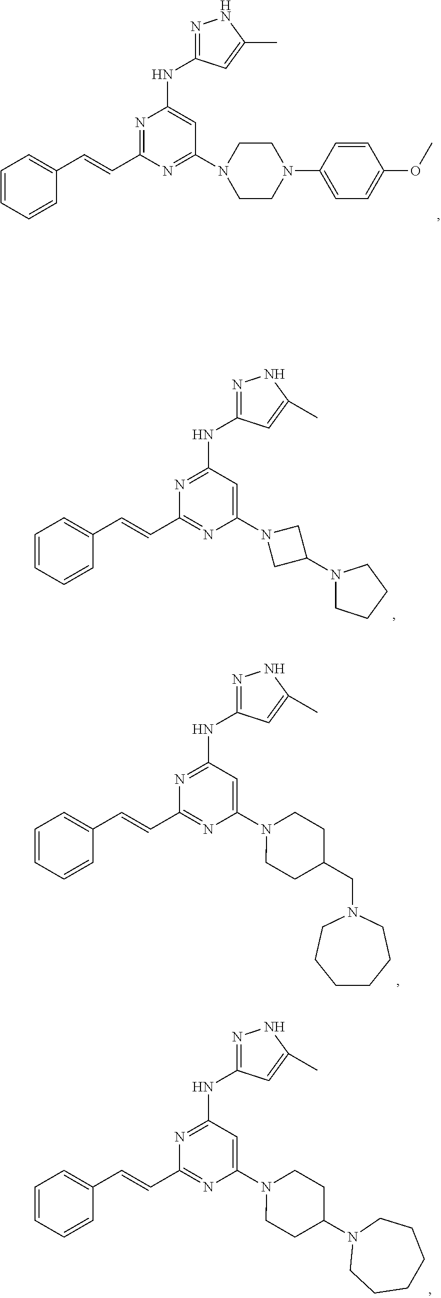 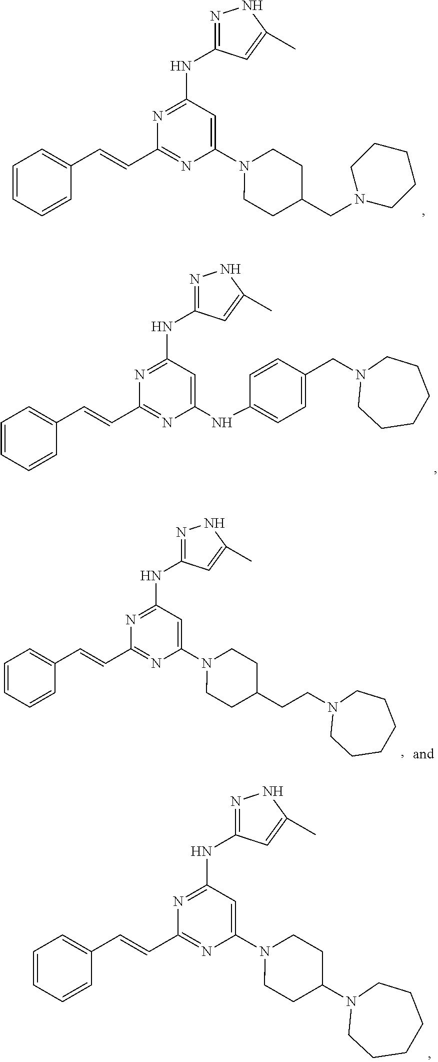
and pharmaceutically acceptable salts or prodrugs thereof.

20. A pharmaceutical composition comprising a compound of claim 1 in combination with a pharmaceutically acceptable carrier, adjuvant or vehicle.

21. The composition of claim 20, wherein the composition comprises particles of the compound that are less than about 2 microns average particle size.

22. The composition of claim 20, wherein the composition comprises a biodegradable or non-biodegradable polymer.

23. The composition of claim 20, comprising a compound selected from claim 1 and an additive.

24. The composition of claim 23, wherein the additive is selected from an anti-oxidant, a buffer, a bacteriostat, a liquid carrier, a solute, a suspending agent, a thickening agent, a flavoring agent, a gelatin, glycerin, a binder, a lubricant, an inert diluent, a preservative, a surface active agent, a dispersing agent, a biodegradable polymer, or any combination thereof.

25. The composition of claim 20, wherein the carrier is suitable for oral, intraveneous, subcutaneous, parenteral, inhalation, topical, or intradermal administration.

26. A method for the treatment of disease selected from the group consisting of an autoimmune disease, inflammatory disease, neurological or neurodegenerative disease, cancer, cardiovascular disease, allergy, asthma, a hormone-related disease, and a disease associated with undesirable neovascularization, comprising administering to a host in need thereof an effective amount of a compound of claim 1, optionally in a pharmaceutically acceptable carrier.

27. The method of claim 26, wherein the disease is cancer.

28. The method of claim 27, wherein the cancer is a solid tumor, blood borne tumor, breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity, pharynx, lip, tongue, mouth, pharynx, small intestine, colon-rectum, large intestine, rectum, brain and central nervous system, or leukemia.

29. The method of claim 26, wherein the disease associated with undesirable neovascularization comprises ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, Scleritis, Steven-Johnson disease, pemphigoid, radial keratotomy, or corneal graph rejection, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Bechet's disease, infections causing a retinitis or choroiditis, presumed ocular histoplasmosis, Best's disease, myopia, optic pits, Stargart's disease, pars planitis, chronic retinal detachment, hyperviscosity syndromes, toxoplasmosis, or post-laser complications.

30. The method of claim 26, wherein the inflammatory disease is excessive or abnormal stimulation of endothelial cells, atherosclerosis, vascular malfunctions, abnormal wound healing, inflammatory and immune disorders, Bechet's disease, gout or gouty arthritis, abnormal angiogenesis accompanying rheumatoid arthritis, skin diseases, psoriasis, diabetic retinopathy, retinopathy of prematurity, retrolental fibroplasia, macular degeneration, corneal graft rejection, neovascular glaucoma or Osler Weber syndrome.

31. The method of claim 26, wherein the compound is administered in the form of a tablet, a capsule, a lozenge, a cachet, a solution, a suspension, an emulsion, a powder, an aerosol, a suppository, a spray, a pastille, an ointment, a cream, a paste, a foam, a gel, a tampon, a pessary, a granule, a bolus, a mouthwash, or a transdermal patch.

32. (canceled)
